(19) United States
(12) Patent Application Publication
(10) Pub. No.: US 2005/0221284 A1
(43) Pub. Date: Oct. 6, 2005

(54) Heteropolymer complexes and methods for their use

(76) Inventors: Ronald P. Taylor, Keswick, VA (US); Maria L. Craig, Shipman, VA (US); Chang S. Hahn, Charlottesville, VA (US)

Correspondence Address: JONES DAY, 222 EAST 41ST ST, NEW YORK, NY, 10017, US

(21) Appl. No.: 10/484,374

(22) PCT Filed: Jul. 17, 2002
(86) PCT No.: PCT/US02/23141

§ 371 Date: Dec. 29, 2004

Related U.S. Application Data
(60) Provisional application No. 60/305,989, filed on Jul. 17, 2001.

(51) Int. Cl. C12Q 1/70; A61K 39/395; C07K 16/28

(52) U.S. Cl. 435/5; 424/144.1; 530/388.22

(57) ABSTRACT

The present invention relates to an improved heteropolymer complex. The improved heteropolymer complex comprises a first monoclonal antibody specific for a C3b-like receptor (known as complement receptor (CR1) or CD35 in primates and Factor H in other mammals, e.g., dog, mouse, rat, pig, rabbit) site chemically crosslinked (covalently linked) to a second monoclonal antibody, in which the isotype of at least the second monoclonal antibody is the isotype having the highest affinity for the Fc receptor, e.g., in humans, IgG1 or IgG3. The present invention also relates to methods for immune clearance of an antigen in a mammal via the C3b-like receptor comprising administering to said mammal an improved heteropolymer complex of the invention. The present invention also relates to methods for treating or preventing viral infection or microbial infection in a mammal comprising administering to said mammal an improved heteropolymer complex of the invention. The present invention also relates to methods for treating or preventing septic shock in a mammal comprising administering to said mammal an improved heteropolymer complex of the invention. The present invention also relates to methods for treating cancer in a mammal comprising administering to said mammal an improved heteropolymer complex of the invention. The present invention further relates to pharmaceutical compositions for the treatment or prevention of viral infection, microbial infection, septic shock, and cancer comprising an improved heteropolymer complex of the invention.

The present application claims priority benefits to U.S. Provisional Patent Application Ser. No. 60/305,989 filed Jul. 17, 2001, the disclosure of which is incorporated herein by reference in its entirety.

### 1. FIELD OF THE INVENTION

The present invention relates to an improved heteropolymer complex. The improved heteropolymer complex comprises a first monoclonal antibody specific for a C3b-like receptor of a mammal (known as complement receptor (CR1) or CD35 in primates and Factor H in other non-primate mammals, e.g., dog, mouse, rat, guinea pig, rabbit) chemically crosslinked (covalently linked) to a second monoclonal antibody, in which the isotype of at least the second monoclonal antibody is the isotype having the highest known affinity for the Fc receptor in said mammal, e.g., in humans, IgG1 or IgG3. The present invention also relates to methods for immune clearance of an antigen in a mammal via the C3b-like receptor comprising administering to said mammal an improved heteropolymer complex of the invention. The present invention also relates to methods for treating or preventing viral infection or microbial infection in a mammal comprising administering to said mammal an improved heteropolymer complex of the invention. The present invention also relates to methods for treating or preventing septic shock in a mammal comprising administering to said mammal an improved heteropolymer complex of the invention, and in which the second monoclonal antibody specifically binds to lipopolysaccharide, an endotoxin or a constituent of the outer wall of a gram negative bacteria. The present invention also relates to methods for treating cancer in a mammal comprising administering to said mammal an improved heteropolymer complex of the invention. The present invention further relates to pharmaceutical compositions for the treatment or prevention of viral infection, microbial infection, cancer, and septic shock comprising an improved heteropolymer complex of the invention.

### 2. BACKGROUND OF THE INVENTION

The immune adherence reaction was first described in 1953 by Nelson, 1953, Science 118:733-737 and provided strong support for a biological role for primate erythrocytes (E) in defense against infectious agents. Nelson reported that opsonization of bacteria with specific antibodies followed by complement activation promoted binding and immobilization of the bacteria on primate E. Once adhered to the E, the immune-complexed bacteria were efficiently transferred to acceptor phagocytic cells in a reaction in which the bacteria were stripped from the E without any discernable damage to the E. Twenty-seven years later Fearon, 1980, J. Exp. Med. 152:20-30 identified and characterized the first complement receptor, CR1, most specific for the complement activation product C3b, which is now known to facilitate the E immune adherence reaction.

The putative role of primate E CR1 in providing a defense against microorganisms, involves the rapid immobilization and capture of bacteria and/or viruses present in the bloodstream on E before the bacteria and/or viruses can invade susceptible organs and tissues and/or adhere to and colonize sites in the vasculature. Moreover, Nelson reported that transfer of the E-bound bacteria to phagocytes occurred efficiently and rapidly in vitro and this reaction was followed by the phagocytosis and degradation of the internalized bacteria. This observation implies that similar reaction would occur in vivo, where the E-bound pathogen would be transferred to acceptor cells such as fixed tissue macrophages in the liver and spleen.

2.1 The Transfer Reaction and CR1

CR1 was first identified based on its ability to down-regulate amplification of the complement cascade and in particular to serve as a cofactor in the Factor I-mediated degradation of activated C3b. Cornacoff et al., 1983, J Clin Invest 71:236-247 reported that primate E CR1 can bind soluble, as well as particulate, complement opsonized immune complexes (IC) in the circulation. In fact, in vitro models of the transfer reaction with soluble IC have often focused on the potential role of Factor I in catalyzing the breakdown of CR1-bound and IC-associated C3b to C3bi and then C3dg, ligands that do not bind to CR1. The degradation of C3b thus releases complement-opsonized IC from E CR1 back into the plasma. This release, which has been shown to be quite rapid in vitro (half-life of ˜5 min) for IC prepared with small soluble proteins, might be expected to play a role in IC clearance and the transfer reaction in vivo. However, extensive kinetic analyses of E-bound IgG antibody/dsDNA IC in plasma demonstrated the marked stability of these complexes in vitro, which is in contrast to their rapid clearance from the circulation of non-human primates. In addition, the work of Emlen et al. demonstrated that in vitro transfer of E-bound IC to human monocytes was independent of Factor I (see Emlen et al., 1989, J Immunol 142:4366-4371 and Emlen et al., 1992, Clin Exp Immunol 89:8-17).

2.2 Immune Complexes (IC)

IC prepared with systemic lupus erythematosus (SLE) IgG anti-dsDNA antibodies and dsDNA of varying lengths provide a particularly useful model for examining the immune adherence reaction. The multivalent nature of dsDNA allows for high avidity IgG binding. This leads to the generation of stable and soluble complexes which activate complement, capture C3b, and then rapidly bind to primate E. There is little crosslinking between dsDNA molecules in these complexes; therefore their properties and ability to interact with the complement system are essentially defined by the relative number and density of IgG bound per dsDNA molecule.

It has been reported that in vitro binding of IgG antibody/dsDNA IC to chimpanzee E is stable in the presence of a source of Factor I, as manifested by less than 10% release after 1 h for a variety of IC prepared with different sizes of dsDNA (Kimberly et al., 1989, J Clin Invest 84:962-970 and Edberg et al., 1992, Eur J Immunol 22:1333-1339). However, when these complexes are labeled with ¹²⁵I, opsonized with complement and bound in vitro to ⁵¹Cr-labeled chimpanzee E and then re-infused into the animal, the E-bound IC are cleared from the circulation with a half-life of only 5 min, less than 2% of the infused material is released into the plasma, and there is virtually no loss of the ⁵¹Cr-labeled E during this process. Thus, the specific properties of E-bound complement-opsonized IgG antibody/dsDNA IC reveal a contradiction: although the complexes are very stable in vitro in plasma containing Factor I, they are rapidly stripped from the E surface and cleared from the circulation in vivo. This kinetic contradiction provides an important clue that, at least for the IgG antibody/dsDNA IC, and presumably for other IC if the results can be generalized, the in vivo transfer reaction is facilitated by a process which is unlikely to depend upon Factor I-mediated release.

2.3 Heteropolymers

Based on the analyses of the dynamics of the in vitro and in vivo binding and in vivo clearance of IgG antibody/dsDNA IC mediated by primate E in the presence of complement, the immune adherence function of primate E was used to develop therapeutic modalities for targeting and clearing pathogens in the bloodstream (see U.S. Pat. Nos. 5,487,890 and 5,879,679, the disclosures of which are incorporated herein). In particular, bispecific monoclonal antibody (mAb) complexes (heteropolymers, HP), comprising a mAb specific for CR1 chemically crosslinked with a mAb specific for a target pathogen, were used to bind and immobilize a pathogen onto CR1 of the primate E.

Several mouse mAbs specific for E CR1 with affinities in excess of 10⁹ M⁻¹ have been generated, and thus through the use of high affinity pathogen specific mAbs, it is possible to bind virtually any target pathogen to E in the absence of complement, including bacteria and viruses. HP-mediated binding can also be enhanced by the simultaneous ligation of several HP to a single pathogen and to a clustered CR1 region on the E. See, Kuhn et al., 1998, J. Immunol. 160:5088; Hahn et al., 2001, J. Immunol. 166:1057; Taylor et al., 1991, Proc. Natl. Acad. Sci. USA 88:3305; Taylor et al., 1997, J. Immunol. 159:4035; Reist et al., 1994, Eur. J. Immunol. 24:2018; Taylor et al., 1997, J. Immunol. 158:842; Nardin et al., 1999, Mol. Immunol. 36:827; and Cornacoff et al., 1983, J. Clin. Invest. 71:236.

2.4 Concerted Loss of CR1 and IC Clearance

Although not intending to be limited to any specific mechanism, FIG. 1 shows a schematic of the proposed mechanism for the transfer reaction. The first step in the transfer reaction involves recognition and engagement of the E-bound IC by Fc receptors on the phagocytic cell. This step should occur for both C3b-opsonized IC as well as for IC bound to E via HP. This binding is followed by a concerted reaction in which CR1 is cleaved by membrane-associated proteases on the acceptor cell (Step 2), and then the entire IC, including CR1, is internalized via Fc receptors of acceptor cells such as Kupffer cells in the liver (Step 3).

A simple in vivo model for the study of complement independent binding of IC to E CR1 in nonhuman primates can be established by intravenous infusion of a mouse anti-CR1 mAb, followed by polyclonal monkey anti-mouse IgG. Infusion of ¹²⁵I-labeled anti-CR1 mouse mAb 7G9 into the circulation of a rhesus monkey resulted in rapid binding of the mAb to E; however, there was little clearance from the circulation over 1 h (FIG. 2A, filled circles). When polyclonal monkey anti-mouse IgG preparation was infused, the infused monkey IgG bound directly to the mouse mAb 7G9 already liganded to CR1 (FIG. 2B, open squares, mouse anti-human IgG; filled diamonds, capture of mouse IgG), and was rapidly removed from the E and cleared from the circulation without loss of the E (i.e., no change in hematocrit). In fact, more than 90% of the E-bound IC (both ¹²⁵I-labeled anti-CR1 as well as the monkey anti-mouse IgG) were removed from the E. Western blot analysis and a RIA with a second non-crossreacting anti-CR1 mAb HB8592, demonstrated that CR1 was also removed from the E at the same rate at which the IC were cleared (FIG. 2A, open circles). A plausible mechanism to explain this concerted reaction would be loss of CR1 by proteolytic cleavage followed by uptake of the released IC by acceptor macrophages. In fact, when anti-CR1 mAb was labeled with ¹³¹I, and imaged, the cleared counts were localized principally to the liver, and to a lesser extent to the spleen.

Citation of a reference in this section or any section of this application shall not be construed as an admission that such reference is prior art to the present invention.

### 3. SUMMARY OF THE INVENTION

The present inventors have surprisingly discovered that the selection of the isotype used for the monoclonal antibody component of a heteropolymer complex can dramatically affect the efficiency of the complex to clear pathogens or immunogens or antigens that are bound to the complement receptor CR1 (CD35) expressed on erythrocytes in primates or a functionally analogous molecule. More particularly, the present inventors have concluded that immune clearance efficiency is dramatically and advantageously enhanced by use of heteropolymer complexes in which at least the second monoclonal antibody is of the isotype having the highest known affinity for the Fc receptor in a particular species, e.g. in humans, IgG1 or IgG3.

The present invention is directed to a heteropolymer complex, comprising a first monoclonal antibody specific for a C3b-like receptor of a mammal chemically crosslinked (covalently linked) to a second monoclonal antibody, in which the isotype of at least the second monoclonal is the isotype having the highest known affinity for the Fc receptor in said mammal. In a preferred embodiment, the first monoclonal antibody is specific for complement receptor (CR1 or CD35) on a human erythrocyte and the isotype of the second monoclonal antibody is human IgG1 or human IgG3. In a preferred aspect of this embodiment, the first monoclonal antibody is a human or humanized monoclonal antibody, preferably having the human IgG1 or human IgG3 isotype. In certain embodiments where the second monoclonal antibody is a human, humanized or chimeric antibody, the antibody has at least equal affinity for the human Fc receptor as a human IgG1 or human IgG3 antibody. Where the first monoclonal antibody is a mouse monoclonal antibody specific for primate CR1, the second monoclonal antibody is not a mouse monoclonal antibody having the isotype IgG2a. In another embodiment, the first monoclonal antibody is specific for Factor H on a non-primate mammalian platelet and the isotype of the second monoclonal antibody is the isotype having the highest known affinity for the Fc receptor in said non-primate mammal. The antigen to which the second monoclonal antibody specifically binds can be a viral, microbial or cancer cell-specific antigen.

In another embodiment, the present invention is directed to a heteropolymer cocktail composition comprising at least two heteropolymer complexes, in which at least one complex comprises a first monoclonal antibody specific for a C3b-like receptor of a mammal chemically crosslinked to a second monoclonal antibody, in which the isotype of at least the second monoclonal antibody is the isotype having the highest known affinity for the Fc receptor in said mammal. In a preferred embodiment, the first monoclonal antibody is specific for complement receptor (CR1 or CD35) on a human erythrocyte and the isotype of the second monoclonal antibody is human IgG1 or human IgG3. In a preferred aspect of this embodiment, the isotype of the second monoclonal antibody in each complex is human IgG1 or human IgG3. In another embodiment, the first monoclonal antibody is specific for Factor H on a non-primate mammalian platelet and the isotype of the second monoclonal antibody is the isotype having the highest known affinity for the Fc receptor in said non-primate mammal.

In another embodiment, the present invention is directed to a method for immune clearance of an antigen comprising administering to a mammal an effective amount of a heteropolymer complex, said complex comprising a first monoclonal antibody specific for a C3b-like receptor of said mammal chemically crosslinked to a second monoclonal antibody, in which the isotype of at least the second monoclonal antibody is the isotype having the highest known affinity for the Fc receptor in said mammal. The method may further comprise allowing said complex to bind to at least one C3b-like receptor site and to said antigen. The method may yet further comprise permitting said bound complex to be cleared from circulation of said mammal. In a preferred embodiment, the first monoclonal antibody is specific for complement receptor (CR1 or CD35) on a human erythrocyte and the isotype of the second monoclonal antibody is human IgG 1 or human IgG3. In another embodiment, the first monoclonal antibody is specific for Factor H on a non-primate mammalian platelet and the isotype of the second monoclonal antibody is the isotype having the highest known affinity for the Fc receptor in said non-primate mammal.

In yet another embodiment, the present invention is directed to a method for immune clearance of an antigen comprising administering to a mammal an effective amount of a heteropolymer complex cocktail comprising at least two complexes, in which at least one complex comprises a first monoclonal antibody specific for a C3b-like receptor of said mammal chemically crosslinked to a second monoclonal antibody, in which the isotype of at least the second monoclonal antibody is the isotype having the highest known affinity for the Fc receptor in said mammal. In a preferred embodiment, the first monoclonal antibody is specific for complement receptor (CR1 or CD35) on a human erythrocyte and the isotype of the second monoclonal antibody is human IgG1 or human IgG3. In a preferred aspect of this embodiment, the isotype of the second monoclonal antibody in each complex is human IgG1 or human IgG3. In another embodiment, the first monoclonal antibody is specific for Factor H on a non-primate mammalian platelet and the isotype of the second monoclonal antibody is the isotype having the highest known affinity for the Fc receptor in said non-primate mammal. The method may further comprise allowing said cocktail to bind to at least one C3b-like receptor site and to said antigen. The method may yet further comprise permitting said bound cocktail to be cleared from circulation of said mammal.

The present invention is also directed to a method for immune clearance of an antigen comprising administering to a mammal an effective amount of franked cells expressing a C3b-like receptor bound to a heteropolymer complex, said complex comprising a first monoclonal antibody specific for the C3b-like receptor of said mammal chemically crosslinked to a second monoclonal antibody, in which the isotype of at least the second monoclonal antibody is the isotype having the highest known affinity for the Fc receptor in said mammal. In a preferred embodiment, the first monoclonal antibody is specific for complement receptor (CR1 or CD35) on a human erythrocyte and the isotype of the second monoclonal antibody is human IgG1 or human IgG3. The method may further comprise permitting the antigen to be cleared from circulation of said mammal.

In yet another embodiment, the present invention is directed to a method of detecting the presence of an antigen in a mammal, said method comprising contacting a sample obtained from the mammal containing cells expressing a C3b-like receptor with a heteropolymer complex, which complex comprises a first monoclonal antibody specific for the C3b-like receptor of said mammal chemically crosslinked to a second monoclonal antibody, in which the isotype of at least the second monoclonal antibody is the isotype having the highest known affinity for the Fc receptor in said mammal, and detecting binding of the antigen in the sample. In one aspect of this embodiment, the detecting step comprises separating the cells from soluble components; and contacting the cells with a labeled secondary antibody specific for the antigen. In a preferred embodiment, the method comprises contacting a human whole blood sample containing erythrocytes with a heteropolymer-complex, which complex comprises a first monoclonal antibody specific for erythrocyte complement receptor CR1 site on a human erythrocyte chemically crosslinked to a second monoclonal antibody, in which the isotype of at least the second monoclonal antibody is human IgG1 or human IgG3, and detecting binding of the antigen.

In yet another embodiment, the present invention is directed to a method for treating or preventing viral infection or microbial infection in a mammal comprising administering to said mammal an effective amount of a heteropolymer complex, said complex comprising a first monoclonal antibody specific for a C3b-like receptor of said mammal chemically crosslinked to a second monoclonal antibody, in which the isotype of at least the second monoclonal antibody is the isotype having the highest known affinity for the Fc receptor in said mammal. In a preferred embodiment, the first monoclonal antibody is specific for complement receptor (CR1 or CD35) on a human erythrocyte and the isotype of the second monoclonal antibody is human IgG1 or human IgG3. In another embodiment, the first monoclonal antibody is specific for Factor H on a non-primate mammalian platelet and the isotype of the second monoclonal antibody is the isotype having the highest known affinity for the Fc receptor in said non-primate mammal.

In yet another embodiment, the present invention provides a method for treating or preventing septic shock in a mammal comprising administering to said mammal an effective amount of a heteropolymer complex, said complex comprising a first monoclonal antibody specific for a C3b-like receptor of said mammal chemically crosslinked to a second monoclonal antibody specific for lipopolysaccharide, endotoxin or a constituent of the outer wall of a Gram-negative bacterium, in which the isotype of at least the second monoclonal antibody is the isotype having the highest known affinity for the Fc receptor in said mammal. In a preferred embodiment, the first monoclonal antibody is specific for complement receptor (CR1 or CD35) on a human erythrocyte and the isotype of the second monoclonal antibody is human IgG1 or human IgG3. In another embodiment, the first monoclonal antibody is specific for Factor H on a non-primate mammalian platelet and the isotype of the second monoclonal antibody is the isotype having the highest known affinity for the Fc receptor in said non-primate mammal.

In yet another embodiment, the present invention is directed to a method for treating cancer in a mammal comprising administering to said mammal an effective amount of a heteropolymer complex, said complex comprising a first monoclonal antibody specific for a C3b-like receptor of said mammal chemically crosslinked to a second monoclonal antibody specific for a cancer cell-specific antigen, in which the isotype of at least the second monoclonal antibody is the isotype having the highest known affinity for the Fc receptor in said mammal. In a preferred embodiment, the first monoclonal antibody is specific for complement receptor (CR1 or CD35) on a human erythrocyte and the isotype of the second monoclonal antibody is human IgG1 or human IgG3. In another embodiment, the first monoclonal antibody is specific for Factor H on a non-primate mammalian platelet and the isotype of the second monoclonal antibody is the isotype having the highest known affinity for the Fc receptor in said non-primate mammal.

In an alternative embodiment to any of the above methods, two or more heteropolymer complexes are administered, in which each first monoclonal antibody in the complex can be specific for the same, or a different epitope on the C3b-like receptor; and ach second monoclonal antibody can be specific for the same or a different epitope on the same antigen, or specific for a different antigen.

3.1 Definitions and Abbreviations

The term “antibody specific for a viral antigen, microbial antigen, or cancer cell-specific antigen” and the like as used herein refer to an antibody that immunospecifically binds to a viral antigen, a microbial antigen or a cancer cell-specific antigen and does not specifically bind to other polypeptides. Antibodies that immunospecifically bind to viral antigens, microbial antigens or cancer cell-specific antigens may have cross-reactivity with other antigens. Preferably, an antibody that immunospecifically binds to a viral antigen, a microbial antigen or a cancer cell-specific antigen does not cross-react with other antigens. Antibodies that immunospecifically bind to viral antigens, microbial antigens or cancer cell-specific antigens can be identified, for example, by immunoassays or other techniques known to those of skill in the art.

As used herein, the term “C3b-like receptor” is understood to mean any mammalian circulatory molecule which has an analogous function to the C3b receptor, for example, CR1 (CD35) in human or non-human primates, or Factor H in non-primate mammals (Alexander et al, 2001, J. Biol. Chem. 276:32129). Illustrative examples of C3b-like receptors include, but are not limited to, CR1 (CD35) of human or non-human primates, and Factor H of non-primate mammals.

The term “fragment” as used herein refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of a viral peptide or polypeptide, a microbial peptide or polypeptide or an antibody that specifically binds to a viral, microbial or cancer cell-specific antigen.

The term “franking” as used herein refers to the ex vivo binding of a heteropolymer complex to a cell expressing a C3b-like receptor, e.g., a primate erythrocyte. The cell-bound heteropolymer complex can then be administered to the mammal. The cells can be obtained from the individual to which the franked complex is to be administered or can be obtained from another suitable donor.

The term “fusion protein” as used herein refers to a polypeptide that comprises an amino acid sequence of an antibody or fragment thereof and an amino acid sequence of a heterologous polypeptide (i.e., an unrelated polypeptide).

The term “host cell” as used herein refers to the particular subject cell transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

The term “immune clearance” as used herein refers to the removal of an antigen from the blood of a mammal by the binding of the antigen to a cell-bound heteropolymer complex and results in the reduction of the antigen in the blood, by at least 5%, at least 10%, at least 15%, at least 20%, at least 25%, at least 30%, at least 40%, at least 50%, at least 60%, at least 75%, at least 80%, at least 90%, at least 95% or at least 99%, of a mammal administered a heteropolymer or heteropolymer cocktail composition of the invention relative to a mammal having a similar concentration of antigen in the blood but not administered the composition.

As used herein, an “isolated” or “purified” material is material that is substantially free of other contaminating material. The language “substantially free” includes preparations which are at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% pure (by dry weight). When the material is produced by chemical synthesis, it is preferably substantially free of chemical precursors or other chemicals, i.e., it is separated from chemical precursors or other chemicals which are involved in the synthesis of the material. In a preferred embodiment, the heteropolymer complexes of the invention are isolated or purified.

In certain embodiments of the invention, an “effective amount” is the amount of a heteropolymer or heteropolymer cocktail composition of the invention that reduces the incidence, the severity, the duration and/or the symptoms associated with viral infection or microbial infection or septic shock in a mammal, e.g., a human or non-human primate. In certain other embodiments of the invention, an “effective amount” is the amount of a composition of the invention that results in a reduction in viral titer or microbial titer by at least 2.5%, at least 5%, at least 10%, at least 15%, at least 25%, at least 35%, at least 45%, at least 50%, at least 75%, at least 85%, by at least 90%, at least 95%, or at least 99% in a mammal administered a composition of the invention relative to the viral titer or microbial titer in a mammal or group of mammals (e.g., two, three, five, ten or more mammals) not administered a composition of the invention.

In certain embodiments of the invention, an “effective amount” is the amount of a heteropolymer or heteropolymer cocktail composition of the invention that reduces the incidence, the severity, the duration and/or the symptoms associated with a cancer in a mammal, e.g., a human or non-human primate. In certain other embodiments, an “effective amount” is the amount of a composition of the invention that results in a reduction of the growth or spread of cancer or number of circulating cancer cells by at least 2.5%, at least 5%, at least 10%, at least 15%, at least 25%, at least 35%, at least 45%, at least 50%, at least 75%, at least 85%, by at least 90%, at least 95%, or at least 99% in a mammal administered a composition of the invention relative to the growth or spread of cancer or number of circulating cancer cells in a mammal or group of mammals (e.g., two, three, five, ten or more mammls) not administered a composition of the invention.

Abbreviations used herein include: IC, immune complex(es); HCT, hematocrit; NHS, normal human serum; CR1, primate E complement receptor; CH50, hemolytic complement activity; HP, heteropolymer; CVF, cobra venom factor; CCS, cell culture supernatant; GFP, green fluorescent protein; C3b, and C3bi, C3dg, the major cleavage fragment and further degradation products of C3, respectively; RT, room temperature; SATA, N-succinimidyl S-acetylthioacetate; sSMCC, sulfosuccinimidyl 4-(N-maleimidomethyl)cyclohexane-1-carboxylate.

### 4. BRIEF DESCRIPTION OF THE FIGURES

FIG. 1. Schematic of the proposed mechanism for the transfer reaction. Step 1: The acceptor cell binds to the E/HP/pathogen IC via Fc recognition. Step 2: Proteases associated with the acceptor cell membrane cut CR1. Step 3: The released IC and associated CR1 are internalized by the acceptor cell. The E are spared during this process of focused phagocytosis.

FIGS. 2A-2B. IC formed in situ on E CR1 are cleared from the circulation. 251-labeled anti-CR1 mAb 7G9 (0.8 mg) was infused iv into the circulation of an immunologically naive rhesus monkey (5.3 kg), and more than 75% of the infused reagent bound to E. At 61.5 min (see arrow) a bolus of monkey antibodies to mouse IgG was infused and blood samples were periodically collected and processed for another 80 min. FIG. 2A. The E pellets were counted to measure mAb 7G9 bound in vivo, and the pellets were also probed with ¹²⁵I-labeled anti-CR1 mAbs 7G9 and HB8592 (initially 2900 and 1140 epitopes per E, respectively) to determine total E CR1. The increase in apparent CR1 epitopes after the infusion of monkey anti-mouse IgG at 61.5 min is due to capture of the mouse mAb probes by the monkey anti-mouse IgG bound to the E. More than 90% of the plasma counts associated with the infused ¹²⁵I-labeled mAb 7G9 were also cleared from the circulation at the end of the experiment. FIG. 2B. The E were also probed with several other reagents to demonstrate: E-bound mAb 7G9 (sheep anti-ms IgG); E-bound monkey anti-mouse IgG (mouse anti-huIgG, mAb HB43, which cross reacts with monkey IgG); E-bound monkey anti-mouse IgG (mouse IgG is captured); E-associated C3b (mouse anti-C3b mAb 7C12).

FIGS. 3A-3B. FIG. 3A. Flow cytometric determinations of the percent binding (mean and SD) of GFP-PAO1 to human E mediated by HP or serum as a function of time and incubation medium at 37° C. In most experiments the results represent the average for independent determinations on sera and E from 6 or more donors. The ratio of E/bacteria was −50 to 1. The difference in % bound for serum-mediated binding at 20 min versus 60 min (68±13% versus 39±19) was statistically significant (p=0.003, paired t-test). FIG. 3B. HP-mediated binding of PAO1 to either human or monkey E in whole blood anti-coagulated in EDTA. The E/bacterium ratio was ≧500/1, and incubations were conducted for 15 min at 37° C.

FIGS. 4A-4D. HP-mediated binding of GFP-PAO1 to E in CVF treated cynomolgus monkeys. FIG. 4A. HP mediates binding of GFP-PAO1 to E in the circulation of a cynomolgus monkey (2A, 4.2 kg) treated with CVF 24 h previously. The CH50 of the monkey was 0 on the experimental day; 480 before CVF treatment. GFP-PAO1 was infused for 160 min at a rate of 4×10⁸ CFU/kg/h (total dose: 1×10⁹ CFU/kg), and HP (9H3×2H4, 140 ug/kg) was infused over 1 min at the 59 min point. Initial/final HCT: 23.8/17.3. FIG. 4B. As in FIG. 4A, except the monkey (4B, 2.7 kg) was infused with 5×10⁸ CFU/kg/h of GFP-PAO1 over 120 min (total dose: 1×10⁹ CFU/kg). The CH50 was 17 on the experimental day; 305 before CVF treatment. At 60 min, HP (9H3×2H4, 78 ug/kg) was infused. At 275 min an additional bolus of 9×10⁸ CFU/kg GFP-PAO1 (denoted B) was infused over a few min. Initial/final HCT: 32.5/24.4. FIG. 4C. As in FIG. 4A, except the monkey (4C, 5.1 kg) was not treated with HP. The CH50 of the monkey was 2 on the experimental day; 232 before CVF treatment. GFP-PAO1 was infused continuously at 4×10⁸ CFU/kg/h over 120 min (total dose: 8×10⁸ CFU/kg), and then a bolus (B) of GFP-PAO1 (8×10⁸ CFU/kg) was infused at 270 min. This monkey had 90 CR1 epitopes/E. Initial/final HCT: 36.0/24.6. FIG. 4D. As in FIG. 4A, except the monkey (4D, 3.5 kg) was treated with HP (9H3×2H4, 117 ug/kg) at t=0 before infusion of bacteria. The CH50 of the monkey was 1 on the experimental day; 394 before CVF treatment. GFP-PAO1 was infused at a rate of 8×10⁸ CFU/kg/h for 1 h (total dose: 8×10⁸ CFU/kg). Initial/final HCT: 43.5/33. The reciprocal titers of IgG anti-PAO1 antibodies in monkeys 4A, 4B, 4C, and 4D, were ND, >100, 14, and 7, respectively. CFU, Whole Blood; CFU, SN; and CFU, Pellet are the CFU's measured in the whole blood, plasma supernatant and E pellet, respectively. Particles, SN and Particles, Pellet are the fluorescent events detected in the plasma supernatant and the E pellet, respectively (see Materials and Methods, infra). The duration of the continuous bacterial infusion is denoted by the double headed horizontal arrow in FIGS. 4-7.

FIGS. 5A-5D. HP-mediated binding of GFP-PAO1 to E in complement-replete monkeys. FIG. 5A. HP mediates binding of GFP-PAO1 to E in the circulation of a complement-replete cynomolgus monkey (5A, 2.3 kg, CH50=136). GFP-PAO1 was infused for 180 min at a rate of 1×10⁹ CFU/kg/h (total dose: 3×10⁹ CFU/kg), and HP (7G9×2H4, 120 ug/kg) was infused at 91 min. A liver biopsy was taken from the animal at 75 min, and therefore hematocrits are not reported. FIG. 5B. As in FIG. 5A, except the rhesus monkey (5B, 8.5 kg, CH50=420) was infused with GFP-PAO1 for 240 min at a rate of 1×10⁹ CFU/kg/h (total dose: 4×10⁹ CFU/kg), and HP (7G9×2H4, 127 ug/kg) was infused at 115 min. Final CH50: 364. Initial/final HCT: 38/33. FIG. 5C. As in FIG. 5A except the cynomolgus monkey (5C, 3.4 kg, CH50=550) was infused with GFP-PAO1 for 2 h at a rate of 3.5×10⁸ CFU/kg/h (total dose: 7×10⁸ CFU/kg). Final CH50: 600. Initial/final HCT: 43.4/36.1. FIG. 5D. Control experiment with anti-PAO1 mAb 2H4 alone to test for binding of GFP-PA01 to E in the circulation of a cynomolgus monkey (5D, 4.8 kg, CH50=212). GFP-PA01 was infused for 210 min at a rate of 1×10⁹ CFU/kg/h (total dose: 3.5×10⁹ CFU/kg), and mAb 2H4 was infused at 91 min (60 ug/kg) followed by HP (7G9×2H4, 120 ug/kg) at 151 min. Final CH50: 177. Initial/final HCT: 34/29. The reciprocal titers of IgG anti-PAO1 in the four monkeys (5A-5D) were, respectively, 20; 40; 33; and 20. See FIG. 4 for symbol definitions.

FIGS. 6A-6B. Handling of GFP-PAO1 in the circulation of two cynomolgus monkeys (6A, 3 kg; 6B, 3.3 kg), one of which (6B) was treated with HP (7G9×2H4, 125 ug/kg) 30 min before infusion of the bacteria. Both monkeys were infused with GFP-PAO1 at a rate of 1-1.2×10⁹ CFU/kg/h for 90 min (total dose: 4A, 1.5×10⁹ CFU/kg; 4B, 1.8×10⁹ CFU/kg). See Table II for data on HCT and CH50. The reciprocal titers of IgG anti-PAO1 in the two monkeys were 5 and 17, respectively. See FIG. 4 for symbol definitions.

FIGS. 7A-7B. Effect of infusion of GFP-PAO1 on the levels of TNF-α and IL-1β in the circulation of two cynomolgus monkeys (7A, 3 kg; 7B, 2.3 kg) one of which (7B) was treated with HP (7G9×2H4, 115 ug/kg) 15 min before the start of the bacterial infusion. In order to control blood pressure in the control (no HP, 7A) monkey, phenylephrine was infused continuously starting at the 15 min point. The HP-treated monkey did not receive phenylephrine until the 2 hour mark.

FIG. 8. HP-mediated clearance of ¹³¹I-labeled ΦX174 from the circulation of a male stump-tail macaque monkey (9 kg, 4500 CR1/E). ¹³¹-labeled ΦX174 (100 μg) was infused at time zero, and HP (480 μg) was infused ˜50 min later, denoted by arrow. Liver counts (open squares, right y-axis) are based on 1 min integrated intensities from Anger camera imaging and are multiplied by an arbitrary factor to allow direct comparison with the other parameters. The counts in the liver 5 min after infusion of ¹³¹I-labeled ΦX174 represent counts spontaneously cleared as well as a steady state level due to the large volume of blood circulating in the liver. Soon after HP infusion, >90% of the counts were bound to E, and this binding was accompanied by a rapid drop in plasma-associated counts. By the end of the experiment, >95% of the counts were cleared from the circulation, and this clearance was accompanied by an increase in counts in the liver. The total number of counts infused (based on Anger camera imaging) was 9900, and after clearance was completed, >50% of the infused counts were positively identified with the liver.

FIG. 9. Infused Fab′ X Fab′ HP does not mediate clearance unless followed by whole IgG anti-ΦX174 mAb. At time zero, 100 μg of ¹³¹I-labeled ΦX174 was infused into the circulation of an 8.7-kg female stump-tail macaque monkey (4500 CR1/E), and the Fab′×Fab′ HP (360 μg) was infused 48 min later (left arrow, top axis). Although a very high level of E binding accompanied infusion of the HP, E-bound counts did not clear from the circulation, and liver counts (right y-axis, see FIG. 8 legend) remained flat. 40 min later (right arrow, top axis) whole anti-ΦX174 mAb 7B7 (500 μg) was infused, and after a 5 min delay, clearance commenced and proceeded rapidly. Whole body background (before infusion) was 2,000 counts, liver background was 130 counts, and a total of 31,000 counts were infused.

FIG. 10. Spontaneous clearance to the liver of ¹³¹I-labeled ΦX174 infused (at time 0) into the circulation of a 5.8 kg male cynomolgus monkey with circulating Abs specific for ΦX174. Soon after infusion, >90% of the total counts (open circles) were bound to E (solid circles). Plasma counts (solid triangles) remained low. Before infusion of ¹³¹I-labeled ΦX174, liver counts (right y-axis, see FIG. 8 legend) were <40 and the total number of background counts was 400. The total number of counts infused (based on Anger camera imaging) was ˜14,000. After clearance was completed, >50% of the infused counts were positively identified with the liver.

FIG. 11. HP-mediated E-binding and clearance of DV in cynomolgus monkeys. DV was infused at a rate of ˜3×10⁹ DV particles per kg per h, and 1 h after termination of DV infusion, a bolus of HP (˜200 μg/kg) was injected. 90 min to 2 h after HP injection, a second DV infusion was performed for 1 h using the same dose. Particles in plasma and E-associated (Cell) DV particles were determined at regular time intervals by RT-PCR. The horizontal bars denote the duration of DV infusion and the arrow shows HP injection. Time 0 represents pre-DV infusion blood samples that denote the detection limit of each experiment. Each point represents a mean value and SD from at least 4 independent RT-PCR quantitation reactions from 3 independent RNA isolations. The HP were prepared by crosslinking anti-CR1 mAb 7G9 with either anti-DV IgG1 mAb 9D12 (panel A) or anti-DV IgG2a mAb 1A1D-2 (Panels B and C). In panel C, the monkey was challenged a third time with DV, starting at ˜the 5 h mark.

### 5. DETAILED DESCRIPTION OF THE INVENTION

5.1 Heteropolymer Complex

The present invention provides a heteropolymer complex, comprising a first monoclonal antibody specific for a C3b-like receptor of a mammal chemically crosslinked (covalently linked) to a second monoclonal antibody, in which the isotype of at least the second monoclonal is the isotype having the highest known affinity for the Fc receptor in said mammal. In a preferred embodiment, the first monoclonal antibody is specific for complement receptor (CR1 or CD35) on a human erythrocyte and the isotype of the second monoclonal antibody is human IgG1 or human IgG3. In a preferred aspect of this embodiment, the first monoclonal antibody is a human or humanized monoclonal antibody, preferably having the human IgG1 or human IgG3 isotype. In certain embodiments where the second monoclonal antibody is a human, humanized or chimeric antibody, the antibody has at least equal affinity for the human Fc receptor as a human IgG1 or human IgG3 antibody. Where the first monoclonal antibody is a mouse monoclonal antibody specific for primate CR1, the second monoclonal antibody is not a mouse monoclonal antibody having the isotype IgG2a. In another embodiment, the first monoclonal antibody is specific for Factor H on a non-primate mammalian platelet and the isotype of the second monoclonal antibody is the isotype having the highest known affinity for the Fc receptor in said non-primate mammal. The antigen to which the second monoclonal antibody specifically binds can be a viral, microbial or cancer cell-specific antigen.

In another embodiment, the present invention provides a heteropolymer cocktail composition comprising at least two heteropolymer complexes, in which at least one complex comprises a first monoclonal antibody specific for a C3b-like receptor of a mammal chemically crosslinked to a second monoclonal antibody, in which the isotype of at least the second monoclonal antibody is the isotype having the highest known affinity for the Fc receptor in said mammal. In a preferred embodiment, the first monoclonal antibody is specific for complement receptor (CR1 or CD35) on a human erythrocyte and the isotype of the second monoclonal antibody is human IgG1 or human IgG3. In a preferred aspect of this embodiment, the isotype of the second monoclonal antibody in each complex is human IgG1 or human IgG3. In another embodiment, the first monoclonal antibody is specific for Factor H on a non-primate mammalian platelet and the isotype of the second monoclonal antibody is the isotype having the highest known affinity for the Fc receptor in said non-primate mammal.

Heteropolymer constructs have been previously described, see U.S. Pat. No. 5,487,890. The present inventors have surprisingly discovered that the selection of the isotype used for the monoclonal antibody component of the heteropolymer can dramatically effect the efficiency of the complex to clear pathogens or immunogens or antigens that are bound to cells via a C3b-like receptor, e.g., bound to primate erythrocytes via CR1. More particularly, the present inventors have concluded that immune clearance efficiency is dramatically and advantageously enhanced by use of heteropolymer complexes in which at least the second monoclonal antibody is of the isotype having the highest affinity for the Fc receptor in a particular mammalian species, e.g., in humans, the IgG1 or IgG3 isotype.

The heteropolymers of the present invention are prepared from monoclonal antibodies which are specific for the C3b-like receptor, e.g., CR1 on a primate erythrocyte or Factor H on certain non-primate mammals, and from monoclonal antibodies specific for a particular antigen and which binds the Fc receptor. The monoclonal antibodies must also be capable of being crosslinked (covalently linked) to each other while retaining binding ability for the C3b-like receptor and to the particular antigen, as well as retaining binding ability of the second monoclonal antibody for the Fc receptor.

Monoclonal antibodies are homogeneous populations of antibodies to a particular antigen (e.g., CR1, a viral antigen, a microbial antigen, a cancer cell-specific antigen, etc.). Monoclonal antibodies (mAb) useful in the present invention can be prepared by using any technique known in the art which provides for the production of antibody molecules by continuous cell lines in culture. These include, but are not limited to, the hybridoma technique originally described by Kohler and Milstein (1975, Nature 256, 495-497), the more recent human B cell hybridoma technique (Kozbor et al., 1983, Immunology Today 4: 72), and the EBV-hybridoma technique (Cole et al., 1985, Monoclonal Antibodies and Cancer Therapy, Alan R. Liss, Inc., pp. 77-96). The hybridoma producing the mAbs of use in this invention may be cultivated in vitro or in vivo.

The monoclonal antibodies which may be used in the compositions ands methods of the invention include, but are not limited to, human monoclonal antibodies or chimeric human-mouse (or other species) monoclonal antibodies. Human monoclonal antibodies may be made by any of numerous techniques known in the art (e.g., Teng et al., 1983, Proc. Natl. Acad. Sci. U.S.A. 80, 7308-7312; Kozbor et al., 1983, Immunology Today 4, 72-79; and Olsson et al., 1982, Meth. Enzymol. 92, 3-16).

Additionally, recombinant antibodies, such as chimeric and humanized monoclonal antibodies, comprising both human and non-human portions, which can be made using standard recombinant DNA techniques, are used for the heteropolymer complexes of the invention. A chimeric antibody is a molecule in which different portions are derived from different animal species, such as those having a variable region derived from a murine monoclonal and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, Bio/Techniques 4:214; U.S. Pat. No. 5,225,539; Jones et al., 1986, Nature 321:552-525; Verhoeyan et al. (1988) Science 239:1534; and Beidler et al., 1988, J. Immunol. 141:4053-4060; each of which is incorporated herein by reference n its entirety.

Completely human antibodies are particularly desirable for therapeutic treatment of human patients. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806; each of which is incorporated herein by reference in its entirety. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Bio/technology 12:899-903).

Such antibodies may be of any immunoglobulin class including IgG, IgM, IgE, IgA, and IgD and any subclass thereof. Antibody isotypes are defined by constant-region determinants that distinguish each heavy-chain class and subclass and each light-chain type and subtype within a species. Each isotype is encoded by a separate constant-region gene, and all members of a species carry the same constant-region genes. Within a species, each normal individual will express all isotypes in their serum. Different species inherit different constant-regions genes and therefore express different isotypes. Therefore, when an antibody from one species is injected into another species, the isotypic determinants will be recognized as foreign, inducing an antibody response to the isotypic determinants on the foreign antibody.

Immunoglobulin G (IgG) the most abundant isotype in serum, constitutes about 80% of the total serum immunoglobulin. The IgG molecule is a monomer consisting of two gamma heavy chains and two kappa or lambda light chains. There are four IgG subclasses in humans, numbered in accordance with their decreasing serum concentrations: IgG1 (9 mg/ml), IgG2 (3 mg/ml), IgG3 (1 mg/ml), and IgG4 (0.5 mg/ml). The four subclasses are encoded by different germ-line CH genes whose DNA sequences are 95% homologous. The structural characteristics that distinguish these subclasses from one another are the size of the hinge region and the number and position of the interchain disulfide bonds between the heavy chains. The subtle amino acid differences between subclasses of IgG affect the biological activity of the molecule. IgG1, IgG3 and IgG4 readily cross the placenta and play an important role in protecting the developing fetus. Several IgG subclasses are activators of the complement system, though their effectiveness varies. The IgG3 subclass is the most effective complement activator, followed by IgG1; IgG2 is relatively inefficient at complement activation, and IgG4 is not able to activate the complement system at all. IgG also functions as an opsonin by binding Fc receptors on phagocytic cells, but there are subclass differences in this function also. In humans, IgG1 and IgG3 bind with a high affinity to Fc receptors. IgG4 has an intermediate affinity, and IgG2 has an extremely low affinity. For a general review of antibodies and their subtypes and classifications, see generally, Janis Kuby, Immunology, 1992, W.H. Freeman & Comapny, New York.

Further, antibody isotypes can be engineered using molecular biology techniques, for example, as described in Reff et al., 1994, Blood 83(2):435.

In a preferred embodiment of the invention, the isotype of the second monoclonal antibody is human IgG1 or human IgG3. In another preferred embodiment, the isotype of both the first monoclonal antibody and the second monoclonal antibody is human IgG1 or human IgG3.

The invention further encompasses the use of bispecific antibodies, which are antibodies that have two different variable regions and bind to two different targets, to prepare the heteropolymer complexes of the invention. These bispecific antibodies are distinct from the bispecific heteropolymer complexes of the invention in that a heteropolymer complex is comprised of two monoclonal antibodies, which antibodies can be bispecific antibodies. According to one embodiment of the present invention, a heteropolymer complex comprises a first bispecific monoclonal antibody specific for two different sites on the C3b-like receptor chemically crosslinked to a second monoclonal antibody, in which the isotype of the second monoclonal antibody is the isotype having the highest affinity for the Fc receptor. In another embodiment of the present invention, a heteropolymer complex comprises a first monoclonal antibody specific for the C3b-like receptor chemically crosslinked to a second bispecific monoclonal antibody specific for two sites or epitopes on a particular antigen or specific for two antigens, in which the isotype of the second bispecific monoclonal antibody is the isotype having the highest affinity for the Fc receptor, e.g., in humans, IgG1 or IgG3. In yet another embodiment, a heteropolymer complex comprises a first bispecific monoclonal antibody specific for two different sites on the C3b-like receptor chemically crosslinked to a second bispecific monoclonal antibody specific for two sites or epitopes on a particular antigen or specific for two antigens, in which the isotype of the second bispecific monoclonal antibody is the isotype having the highest affinity for the Fc receptor in a particular mammal.

Methods for making bispecific antibodies are known in the art. Traditional production of full length bispecific antibodies is based on the coexpression of two immunoglobulin heavy chain-light chain pairs, where the two chains have different specificities (Milstein et al., 1983, Nature 305:537-539). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low.

Similar procedures are disclosed in PCT Publication No. WO 93/08829, published 13 May 1993, and in Traunecker et al., 1991, EMBO J. 10:3655-3659.

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in PCT Publication No. WO 94/04690 published Mar. 3, 1994, which is incorporated herein by reference in its entirety. For further details of generating bispecific antibodies see, for example, Suresh et al., Methods in Enzymology, 1986, 121:210.

The invention also provides heteropolymer complexes in which the first monoclonal antibody is specific to a site on the C3b-like receptor is a functionally active fragment, derivative or analog of an antibody. Functionally active means that the fragment, derivative or analog is able to elicit anti-anti-idiotype antibodies that recognize the same antigen that the antibody from which the fragment, derivative or analog is derived recognized. Specifically, in a preferred embodiment the antigenicity of the idiotype of the immunoglobulin molecule may be enhanced by deletion of framework and CDR sequences that are C-terminal to the CDR sequence that specifically recognizes the C3b-like receptor. To determine which CDR sequences bind the C3b-like receptor, synthetic peptides containing the CDR sequences can be used in binding assays with the C3b-like receptor by any binding assay method known in the art (e.g., the BIA core assay).

Other embodiments of the invention include heteropolymer complexes in which fragments of the antibodies specific for the C3b-like receptor, e.g., CR1, include, but are not limited to, F(ab′)2 fragments, which contain the variable region, the light chain constant region and the CH1 domain of the heavy chain can be produced by pepsin digestion of the antibody molecule, and Fab fragments, which can be generated by reducing the disulfide bridges of the F(ab′)₂ fragments. The invention also provides heavy chain and light chain dimers of the antibody specific for the C3b-like receptor, or any minimal fragment thereof, such as Fvs or single chain antibodies (SCAs) (e.g., as described in U.S. Pat. No. 4,946,778; Bird, 1988, Science 242:423-42; Huston et al., 1988, Proc. Natl. Acad. Sci. USA 85:5879-5883; and Ward et al., 1989, Nature 334:544-54).

In other embodiments, the heteropolymer complexes of the invention are prepared using fusion proteins of an antibody (or functionally active fragments thereof). For example, the first, second or both monoclonal antibody is fused via a covalent bond (e.g., a peptide bond), at either the N-terminus or the C-terminus to an amino acid sequence of another protein (or portion thereof, preferably at least 10, 20 or 50 amino acid portion of the protein) that is not the antibody and such that binding affinity for the Fc receptor is unaffected. Preferably, the antibody or fragment thereof is covalently linked to the other protein at the N-terminus of the constant domain.

The heteropolymer complex antibodies include analogs and derivatives that are either modified, i.e, by the covalent attachment of any type of molecule as long as such covalent attachment does not prevent the antibody from immunospecifically binding the epitope for which it is specific. For example, but not by way of limitation, the derivatives and analogs of the antibodies include those that have been further modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the analog or derivative may contain one or more non-classical amino acids.

The heteropolymer complex antibodies of the invention include antibodies with modifications (e.g., substitutions, deletions or additions) in amino acid residues that interact with Fc receptors. In particular, the antibodies of the invention include antibodies with modifications in amino acid residues identified as involved in the interaction between the Fc domain and the Fc receptor (see, e.g., PCT Publication No. WO 97/34631, which is incorporated herein by reference in its entirety).

In the production of antibodies, screening for the desired antibody can be accomplished by techniques known in the art (e.g., enzyme-linked immunosorbent assay or ELISA). For example, to select antibodies which recognize a specific domain of a pathogen protein, one may assay generated hybridomas for a product which binds to a fragment containing such domain.

For selection of an antibody that specifically binds a first pathogen but which does not specifically bind a different pathogen, one can select on the basis of positive binding to the first pathogen and a lack of binding to the second pathogen.

5.2 Antigens

Any viral, microbial or cancer cell-specific antigen can be used to obtain the second monoclonal antibody to prepare the heteropolymer complexes of the invention. Preferably, antibodies immunospecific for a viral antigen or microbial antigen which are administered to humans are humanized or human monoclonal antibodies. More preferably, the isotype of the second monoclonal antibody is human IgG1 or human IgG3. As used herein, the term “viral antigen” includes, but is not limited to, any viral peptide, polypeptide, protein, which is capable of eliciting an immune response. Illustrative examples of viral antigens are antigens of retroviruses (e.g., human T-cell lymphotrophic virus (HTLV) types I and II and human immunodeficiency virus (HIV)), herpes viruses (e.g., herpes simplex virus (HSV) types I and II, Epstein-Barr virus and cytomegalovirus), arenaviruses (e.g., lassa fever virus), parvoviruses, paramyxoviruses (e.g., morbillivirus virus, human respiratory syncytial virus, and pneumovirus), arboviruses, adenoviruses, bunyaviruses (e.g., hantavirus), cornaviruses, filoviruses (e.g., Ebola virus), flaviviruses (e.g., hepatitis C virus (HCV), yellow fever virus, and Japanese encephalitis virus), hepadnaviruses (e.g., hepatitis B viruses (HBV)), orthomyoviruses (e.g., Sendai virus and influenza viruses A, B and C), papovaviruses (e.g., papillomavirues), picomaviruses (e.g., rhinoviruses, enteroviruses and hepatitis A viruses), poxviruses, reoviruses (e.g., rotavirues), togaviruses (e.g., rubella virus), and rhabdoviruses (e.g., rabies virus). Specific viral antigens include HIV gp120, HIV nef, RSV F glycoprotein, influenza virus neuraminidase, influenza virus hemagglutinin, HTLV tax, herpes simplex virus glycoprotein (e.g., gB, gC, gD, and gE) and hepatitis B surface antigen.

As used herein, the term “microbial antigen” includes, but is not limited to, any microbial peptide, polypeptide, protein, saccharide, polysaccharide, or lipid molecule (e.g., a bacterial, fungi, pathogenic protozoa, or yeast polypeptide including, e.g., LPS and capsular polysaccharide 5/8) which is capable of eliciting an immune response. Further illustrative microbial antigens are antigens of Streptococcus sp., Neisseria sp., Corynebacterium sp., Clostridium sp., Haemophilus sp., Klebsiella sp., Staphylococcus sp., Vibrio sp., Escherichia sp., Pseudomonas sp., Campylobacter (Vibrio) sp., Aeromonas sp., Bacillus sp., Edwardsiella sp., Yersinia sp., Shigella sp., Salmonella sp., Treponema sp., Borrelia sp., Leptospira sp., Mycobacterium sp., Toxoplasma sp., Pneumocystis sp., Francisella sp., Brucella sp., Mycoplasma sp., Rickettsia sp., Chlamydia sp., or Helicobacter sp. Illustrative microbial species include Streptococcus pyogenes, Streptococcus pneumoniae, Neisseria gonorrhoea, Neisseria meningitidis, Corynebacterium diphtheriae, Clostridium botulinum, Clostridium perfringens, Clostridium tetani, Haemophilus influenzae, Klebsiella pneumoniae, Klebsiella ozaenae, Klebsiella rhinoscleromotis, Staphylococcus aureus, Vibrio cholerae, Escherichia coli, Pseudomonas aeruginosa, Campylobacter (Vibrio) jejuni, Campylobacter (Vibrio) fetus, Aeromonas hydrophila, Bacillus cereus, Edwardsiella tarda, Yersinia enterocolitica, Yersinia pestis, Yersinia pseudotuberculosis, Shigella dysenteriae, Shigella flexneri, Shigella sonnei, Salmonella typhimurium, Treponema pallidum, Treponema pertenue, Treponema carateneum, Borrelia vincentii, Borrelia burgdorferi, Leptospira icterohemorrhagiae, Mycobacterium tuberculosis, Toxoplasma gondii, Pneumocystis carinii, Francisella tularensis, Brucella abortus, Brucella suis, Brucella melitensis, Mycoplasma spp., Rickettsia prowazeki, Rickettsia tsutsugumushi, Chlamydia spp., Helicobacter pylori, Coccidioides immitis, Aspergillus fumigatus, Candida albicans, Blastomyces dermatitidis, Cryptococcus neoformans, Histoplasma capsulatum, Entomoeba histolytica, Trichomonas tenas, Trichomonas hominis, Trichomonas vaginalis, Trypanosoma gambiense, Trypanosoma rhodesiense, Trypanosoma cruzi, Leishmania donovani, Leishmania tropica, Leishmania braziliensis, Pneumocystis pneumonia, Plasmodium vivax, Plasmodium falciparum, Plasmodium malaria, Enterobius vermicularis, Trichuris trichiura, Ascaris lumbricoides, Trichinella spiralis, Strongyloides stercoralis, Schistosoma japonicum, Schistosoma mansoni, Schistosoma haematobium, and hookworms.

As used herein, the term “cancer cell-specific antigen” refers to an antigen (e.g., a protein, glycoprotein, polypeptide, peptide, or glycolipid) that is preferentially or differentially expressed on cancer cells relative to non-cancerous cells, preferably normal cells. Examples of cancer cell-specific antigens include, but are not limited, to improperly glycosylated proteins and lipids, CD20, Her-2, and PSMA. Cancer cell-specific antigen also includes a human complement component bound to a cancer cell, e.g., C3b or C3bi bound to a cancer cell, but not C3d or C3g.

Illustrative examples of CR1-specific monoclonal antibodies useful in the present invention include but are not limited to 1B4, HB8592, and 7G9. HB8592 and 1B4 are disclosed in Taylor et al., 1991, Proc. Natl. Acad. Sci., USA 88:3305-3309 and Reist et al., 1993, Eur. J. Immunol. 23:3021-3027. Monoclonal antibody 7G9 is a mAb developed in the present inventors' laboratory and is also disclosed in Reinagel and Taylor, 2000, J. Immunol. 164:1977. Other mAbs to CR1 available and useful include 3D9, E-11, 57F and YZ1 (see, Hogg et al., 1984, Eur. J. Immunol. 14:236; O'Shea et al., 1985, J. Immunol. 134:2580; Nussenzweig, 1982, J. Exp. Med. 151:1427-1438; and Fearon, 1985, J. Immunol. 134:185). Any monoclonal antibody specific for CR1 can be used in the heteropolymers of the present invention. See also, International Patent Publication WO 01/80883 for illustrative methods for the production of anti-C3b-like receptor antibodies.

Illustrative examples of monoclonal antibodies specific for an antigen useful in the present invention include but are not limited to Synagis® (MedImmune, Inc., MD) which is a humanized anti-respiratory syncytial virus (RSV) monoclonal antibody for the treatment of patients with RSV infection; PRO542 (Progenics) which is a CD4 fusion antibody for the treatment of HIV infection; Ostavir (Protein Design Labs, Inc., CA) which is a human antibody for the treatment of hepatitis B virus; Protovir (Protein Design Labs, Inc., CA) which is a humanized IgG, antibody for the treatment of cytomegalovirus (CMV); and anti-LPS antibodies.

Illustrative examples of cancer cell-specific antibodies available for the treatment of cancer include, but are not limited to, Herceptin® (Trastuzumab; Genetech, CA) which is a humanized anti-HER2 monoclonal antibody for the treatment of patients with metastatic breast cancer; Retuxan® (rituximab; Genentech) which is a chimeric anti-CD20 monoclonal antibody for the treatment of patients with non-Hodgkin's lymphoma; IMC-C225 (Imclone Systems Inc., NY) which is a chimeric IgG antibody for the treatment of head and neck cancer; Vitaxin (MedImmune, Inc., MD) which is a humanized antibody for the treatment of sarcoma; Campath I/H (Leukosite, MA) which is a humanized IgG 1 antibody for the treatment of chronic lymphocytic leukemia (CLL); Smart MI95 (Protein Design Labs, Inc., CA) which is a humanized IgG antibody for the treatment of acute myeloid leukemia (AML); LymphoCide (Immunomedics, Inc., NJ) which is a humanized IgG antibody for the treatment of non-Hodgkin's lymphoma; and Smart I D10 (Protein Design Labs, Inc., CA) which is a humanized antibody for the treatment of non-Hodgkin's lymphoma.

5.3 Crosslinking

Once the monoclonal antibodies have been developed, they are crosslinked to form the heteropolymer complex. The chemistry of cross-linking and effective reagents for such purposes are well known in the art. The nature of the crosslinking reagent used to conjugate the monoclonal antibodies is not restricted by the invention. Any crosslinking agent may be used provided that a) the activity (binding ability) of the antibody is retained, and b) binding by the Fc receptor of the Fc portion of at least the second monoclonal antibody is not adversely affected.

An example of an effective crosslinking of monoclonal antibodies is oxidation of Fc with sodium periodate in sodium phosphate buffer for 30 minutes at room temperature, followed by overnight incubation at 4° C. with the second antibody. Conjugation also may be performed by derivatizing one or both monoclonal antibodies with suffosuccinimidyl 6-[3-(2-pyridyldithio)propionamidel hexanoate (sulfo-LC-SPDP, Pierce) for 18 hours at room temperature. For details as to this procedure, see, e.g., Karpovsky et al, 1984, J. Exp. Med. 160:1686-1701; Perez et al, 1985, Nature 316:354-356 or Titus et al, 1987, Journal of Immunology 139:3153-3158. Other procedures are known to those of ordinary skill in the art, and include the procedure set forth by Segal et al., 1995, Curr. Prot. Immnol. 2:131. Conjugates also may be prepared by derivatizing Fc fragments with different crosslinking reagents that will subsequently form a covalent linkage. An example of this reaction is derivatization of Fc fragments with sulfosuccinimidyl 4-(N-maleimidomethyl)cyclohexane-1-carboxylate (Sulfo-SMCC, Pierce) and the second monoclonal antibody is thiolated with N-succinimidyl S-acetylthioacetate (SATA). The derivatized components are purified free of crosslinker and combined at room temperature for one hour to allow crosslinking. Other crosslinking reagents comprising aldehyde, imide, cyano, halogen, carboxyl, activated carboxyl, anhydride and maleimide functional groups are known to persons of ordinary skill in the art and also may be used for conjugation of the monoclonal antibodies. The choice of cross-linking reagent will, of course, depend on the nature of the monoclonal antibodies. The crosslinking reagents described above are effective for protein-protein conjugations. If the compound to be conjugated is a carbohydrate or has a carbohydrate moiety, then heterobifunctional crosslinking reagents such as ABH, M2C2H, MPBH and PDPH are useful for conjugation with a monoclonal antibody (Pierce Chemical Co., Rockford, Ill.). Another method of conjugating proteins and carbohydrates is disclosed by Brumeanu et al. (Genetic Engineering News, Oct. 1, 1995, p. 16).

In all of the above crosslinking reactions it is important to purify the derivatized compounds free of crosslinking reagent. It is important also to purify the final conjugate substantially free of unconjugated reactants. Purification may be achieved by affinity, gel filtration or ion exchange chromatography based on the properties of either component of the conjugate. A particularly preferred method is an initial affinity purification step using protein A-Sepharose to retain Fc and Fc-compound conjugates, followed by gel filtration or ion exchange chromatography based on the mass, size or charge of the Fc conjugate. The initial step of this purification scheme ensures that the conjugate will bind to Fc receptor which is an essential requirement of the invention.

5.4 Therapeutic and Prophylactic Uses of the Heteropolymer Complexes

In another embodiment, the present invention is directed to a method for immune clearance of an antigen comprising administering to a mammal an effective amount of a heteropolymer complex, said complex comprising a first monoclonal antibody specific for a C3b-like receptor of said mammal chemically crosslinked to a second monoclonal antibody, in which the isotype of at least the second monoclonal antibody is the isotype having the highest known affinity for the Fc receptor in said mammal. The method may further comprise allowing said complex to bind to at least one C3b-like receptor site and to said antigen. The method may yet further comprise permitting said bound complex to be cleared from circulation of said mammal. In a preferred embodiment, the first monoclonal antibody is specific for complement receptor (CR1 or CD35) on a human erythrocyte and the isotype of the second monoclonal antibody is human IgG1 or human IgG3. In another embodiment, the first monoclonal antibody is specific for Factor H on a non-primate mammalian platelet and the isotype of the second monoclonal antibody is the isotype having the highest known affinity for the Fc receptor in said non-primate mammal.

In yet another embodiment, the present invention is directed to a method for immune clearance of an antigen comprising administering to a mammal an effective amount of a heteropolymer complex cocktail comprising at least two complexes, in which at least one complex comprises a first monoclonal antibody specific for a C3b-like receptor of said mammal chemically crosslinked to a second monoclonal antibody, in which the isotype of at least the second monoclonal antibody is the isotype having the highest known affinity for the Fc receptor in said mammal. In a preferred embodiment, the first monoclonal antibody is specific for complement receptor (CR1 or CD35) on a human erythrocyte and the isotype of the second monoclonal antibody is human IgG1 or human IgG3. In a preferred aspect of this embodiment, the isotype of the second monoclonal antibody in each complex is human IgG1 or human IgG3. In another embodiment, the first monoclonal antibody is specific for Factor H on a non-primate mammalian platelet and the isotype of the second monoclonal antibody is the isotype having the highest known affinity for the Fc receptor in said non-primate mammal. The method may further comprise allowing said cocktail to bind to at least one C3b-like receptor site and to said antigen. The method may yet further comprise permitting said bound cocktail to be cleared from circulation of said mammal.

The present invention is also directed to a method for immune clearance of an antigen comprising administering to a mammal an effective amount of franked cells expressing a C3b-like receptor bound to a heteropolymer complex, said complex comprising a first monoclonal antibody specific for the C3b-like receptor of said mammal chemically crosslinked to a second monoclonal antibody, in which the isotype of at least the second monoclonal antibody is the isotype having the highest known affinity for the Fc receptor in said mammal. In a preferred embodiment, the first monoclonal antibody is specific for complement receptor (CR1 or CD35) on a human erythrocyte and the isotype of the second monoclonal antibody is human IgG1 or human IgG3. The method may further comprise permitting the antigen to be cleared from circulation of said mammal.

In yet another embodiment, the present invention is directed to a method of detecting the presence of an antigen in a mammal, said method comprising contacting a sample obtained from the mammal containing cells expressing a C3b-like receptor with a heteropolymer complex, which complex comprises a first monoclonal antibody specific for the C3b-like receptor of said mammal chemically crosslinked to a second monoclonal antibody, in which the isotype of at least the second monoclonal antibody is the isotype having the highest known affinity for the Fc receptor in said mammal, and detecting binding of the antigen in the sample. In one aspect of this embodiment, the detecting step comprises separating the cells from soluble components; and contacting the cells with a labeled secondary antibody specific for the antigen. In a preferred embodiment, the method comprises contacting a human whole blood sample containing erythrocytes with a heteropolymer complex, which complex comprises a first monoclonal antibody specific for erythrocyte complement receptor CR1 site on a human erythrocyte chemically crosslinked to a second monoclonal antibody, in which the isotype of at least the second monoclonal antibody is human IgG1 or human IgG3, and detecting binding of the antigen.

In yet another embodiment, the present invention is directed to a method for treating or preventing viral infection or microbial infection in a mammal comprising administering to said mammal an effective amount of a heteropolymer complex, said complex comprising a first monoclonal antibody specific for a C3b-like receptor of said mammal chemically crosslinked to a second monoclonal antibody, in which the isotype of at least the second monoclonal antibody is the isotype having the highest known affinity for the Fc receptor in said mammal. In a preferred embodiment, the first monoclonal antibody is specific for complement receptor (CR1 or CD35) on a human erythrocyte and the isotype of the second monoclonal antibody is human IgG1 or human IgG3. In another embodiment, the first monoclonal antibody is specific for Factor H on a non-primate mammalian platelet and the isotype of the second monoclonal antibody is the isotype having the highest known affinity for the Fc receptor in said non-primate mammal.

Illustrative examples of viral infections which can be treated or prevented in accordance with this invention include, but are limited to, viral infections caused by retroviruses (e.g., human T-cell lymphotrophic virus (HTLV) types I and II and human immunodeficiency virus (HIV)), herpes viruses (e.g., herpes simplex virus (HSV) types I and II, Epstein-Barr virus and cytomegalovirus), arenaviruses (e.g., lassa fever virus), parvoviruses, paramyxoviruses (e.g., morbillivirus virus, human respiratory syncytial virus, and pneumovirus), arboviruses, adenoviruses, bunyaviruses (e.g., hantavirus), cornaviruses, filoviruses (e.g., Ebola virus), flaviviruses (e.g., hepatitis C virus (HCV), yellow fever virus, and Japanese encephalitis virus), hepadnaviruses (e.g., hepatitis B viruses (HBV)), orthomyoviruses (e.g., Sendai virus and influenza viruses A, B and C), papovaviruses (e.g., papillomavirues), picornaviruses (e.g., rhinoviruses, enteroviruses and hepatitis A viruses), poxviruses, reoviruses (e.g., rotavirues), togaviruses (e.g., rubella virus), and rhabdoviruses (e.g., rabies virus). The treatment and/or prevention of a viral infection includes, but is not limited to, alleviating one or more symptoms associated with said infection, the inhibition, reduction or suppression of viral replication, and/or the enhancement of the immune response.

In certain embodiments, a heteropolymer complex or heteropolymer cocktail composition of the invention is administered to a mammal to ameliorate one or more symptoms associated with a viral infection or a disease or disorder resulting, directly or indirectly, from a viral infection. In a specific embodiment, a composition of the invention is administered to a human to ameliorate one or more symptoms associated with AIDS. In certain other embodiments, a composition of the invention is administered to reduce the titer of a virus in a human or non-human primate.

The heteropolymer complex or heteropolymer cocktail of the invention may be administered alone or in combination with other types of anti-viral agents. Examples of anti-viral agents include, but are not limited to: cytokines (e.g., IFN-α, IFN-β, and IFN-γ); inhibitors of reverse transcriptase (e.g., AZT, 3TC, D4T, ddC, ddI, d4T, 3TC, adefovir, efavirenz, delavirdine, nevirapine, abacavir, and other dideoxynucleosides or dideoxyfluoronucleosides); inhibitors of viral mRNA capping, such as ribavirin; inhibitors of proteases such HIV protease inhibitors (e.g., amprenavir, indinavir, nelfinavir, ritonavir, and saquinavir); amphotericin B; castanospermine as an inhibitor of glycoprotein processing; inhibitors of neuramimidase such as influenza virus neuramimidase inhibitors (e.g., zanamivir and oseltamivir); topoisomerase I inhibitors (e.g., camptothecins and analogs thereof); amantadine; and rimantadine. Such anti-viral agents may be administered to a mammal, preferably a non-human primate, more preferably a human, for the prevention or treatment of a viral infection prior to (e.g., 1 minute, 15 minutes, 30 minutes, 45 minutes, 1 hour, 2 hours, 4 hours, 6 hours, 8 hours, 12 hours, 24 hours, 2 days, or 1 week before), subsequent to (e.g., 1 minute, 15 minutes, 30 minutes, 45 minutes, 1 hour, 2 hours, 4 hours, 6 hours, 8 hours, 12 hours, 24 hours, 2 days, or 1 week after), or concomitantly with the administration of a heteropolymer or heteropolymer cocktail composition of the invention.

Illustrative examples of microbial infections which can be treated or prevented in accordance with this invention include, but are not limited to, yeast infections, fungal infections, protozoan infections and bacterial infections. Illustrative organisms that cause microbial infections include, but are not limited to, Streptococcus pyogenes, Streptococcus pneumoniae, Neisseria gonorrhoea, Neisseria meningitidis, Corynebacterium diphtheriae, Clostridium botulinum, Clostridium perfringens, Clostridium tetani, Haemophilus influenzae, Klebsiella pneumoniae, Klebsiella ozaenae, Klebsiella rhinoscleromotis, Staphylococcus aureus, Vibrio cholerae, Escherichia coli, Pseudomonas aeruginosa, Campylobacter (Vibrio) jejuni, Aeromonas hydrophila, Bacillus cereus, Edwardsiella tarda, Yersinia enterocolilica, Yersinia pestis, Yersinia pseudotuberculosis, Shigella dysenteriae, Shigella flexneri, Shigella sonnei, Salmonella typhimurium, Treponema pallidum, Treponema pertenue, Treponema carateneum, Borrelia vincentii, Borrelia burgdorferi, Leptospira icterohemorrhagiae, Mycobacterium tuberculosis, Toxoplasma gondii, Pneumocystis carinii, Francisella tularensis, Brucella abortus, Brucella suis, Brucella melitensis, Mycoplasma spp., Rickettsia prowazeki, Rickettsia tsutsugumushi, Chlamydia spp., Helicobacter pylori, Coccidioides immitis, Aspergillus fumigatus, Candida albicans, Blastomyces dermatitidis, Cryptococcus neoformans, Histoplasma capsulatum, Entomoeba histolytica, Trichomonas tenas, Trichomonas hominis, Trichomonas vaginalis, Trypanosoma gambiense, Trypanosoma rhodesiense, Trypanosoma cruzi, Leishmania donovani, Leishmania tropica, Leishmania braziliensis, Pneumocystis pneumonia, Plasmodium vivax, Plasmodium falciparum, Plasmodium malaria, Enterobius vermicularis, Trichuris trichiura, Ascaris lumbricoides, Trichinella spiralis, Strongyloides stercoralis, Schistosoma japonicum, Schistosoma mansoni, Schistosoma haematobium, and hookworms. The treatment and/or prevention of a microbial infection includes, but is not limited to, alleviating one or more symptoms associated with said infection, the inhibition, reduction or suppression of microbial replication, and/or the enhancement of the immune response.

In certain embodiments, a heteropolymer complex composition of the invention is administered to a mammal, preferably to a non-human primate, more preferably to a human to ameliorate one or more symptoms associated with a microbial infection or a disease or disorder resulting, directly or indirectly, from a microbial infection. In certain other embodiments, a composition of the invention is administered to reduce the number or microbes in a mammal.

The heteropolymer complex or heteropolymer cocktail of the invention may be administered alone or in combination with other types of anti-microbial agents. Illustrative examples of anti-microbial agents include, but are not limited to: antibiotics such as penicillin, amoxicillin, ampicillin, carbenicillin, ticarcillin, piperacillin, cepalospolin, vancomycin, tetracycline, erythromycin, amphotericin B, nystatin, metronidazole, ketoconazole, and pentamidine. Such anti-microbial agents may be administered to a human or non-human primate, preferably a human, for the prevention or treatment of a microbial infection prior to (e.g., 1 minute, 15 minutes, 30 minutes, 45 minutes, 1 hour, 2 hours, 4 hours, 6 hours, 8 hours, 12 hours, 24 hours, 2 days, or 1 week before), subsequent to (e.g., 1 minute, 15 minutes, 30 minutes, 45 minutes, 1 hour, 2 hours, 4 hours, 6 hours, 8 hours, 12 hours, 24 hours, 2 days, or 1 week after), or concomitantly with the administration of the heteropolymer complex. In certain embodiments, primates with increased risk of a viral or bacterial infection are administered a composition of the invention. Illustrative examples of patient populations include, but are not limited to, human burn patients, infants (18 months old or less), immunocompromised or immunodeficient humans, and the elderly (greater than 60 years old).

In yet another embodiment, the present invention provides a method for treating or preventing septic shock in a mammal comprising administering to said mammal an effective amount of a heteropolymer complex, said complex comprising a first monoclonal antibody specific for a C3b-like receptor of said mammal chemically crosslinked to a second monoclonal antibody specific for lipopolysaccharide, endotoxin or a constituent of the outer wall of a Gram-negative bacterium, in which the isotype of at least the second monoclonal antibody is the isotype having the highest known affinity for the Fc receptor in said mammal. In a preferred embodiment, the first monoclonal antibody is specific for complement receptor (CR1 or CD35) on a human erythrocyte and the isotype of the second monoclonal antibody is human IgG1 or human IgG3. In another embodiment, the first monoclonal antibody is specific for Factor H on a non-primate mammalian platelet and the isotype of the second monoclonal antibody is the isotype having the highest known affinity for the Fc receptor in said non-primate mammal. In certain embodiments, mammals with increased risk of septic shock are administered a composition of the invention. Examples of such mammals include, but are not limited to human burn patients, infants (18 months old or less), immunocompromised or immunodeficient humans, and the elderly (greater than 60 years old).

In another specific embodiment, an effective amount of a heteropolymer complex of the invention is administered to an animal in order to ameliorate one or more symptoms associated with septic shock.

The heteropolymer complex or heteropolymer cocktail of the invention may be administered alone or in combination with any other known technique for the treatment or prevention of septic shock in said mammal. Examples of known techniques for the treatment or prevention of septic shock include, but are not limited to, antithrombin, intravenous immunoglobulins, cytokine antagonists (e.g., anti-tumor necrosis factor (TNF) antibodies, soluble TNF receptor, anti-interleukin-1 (IL-1) antibodies, and soluble IL-1 receptor), antibiotics, and anti-inflammatory agents. The treatment and/or prevention of septic shock includes, but is not limited to, alleviating one or more symptoms associated with one or more symptoms with septic shock and the enhancement of the immune response. Such additional known techniques may be administered to a mammal, preferably a human, for the prevention or treatment of septic shock prior to (e.g., 1 minute, 15 minutes, 30 minutes, 45 minutes, 1 hour, 2 hours, 4 hours, 6 hours, 8 hours, 12 hours, 24 hours, 2 days, or 1 week before), subsequent to (e.g., 1 minute, 15 minutes, 30 minutes, 45 minutes, 1 hour, 2 hours, 4 hours, 6 hours, 8 hours, 12 hours, 24 hours, 2 days, or 1 week after), or concomitantly with the administration of the heteropolymer complex.

In yet another embodiment, the present invention is directed to a method for treating cancer in a mammal comprising administering to said mammal an effective amount of a heteropolymer complex, said complex comprising a first monoclonal antibody specific for a C3b-like receptor of said mammal chemically crosslinked to a second monoclonal antibody specific for a cancer cell-specific antigen, in which the isotype of at least the second monoclonal antibody is the isotype having the highest known affinity for the Fc receptor in said mammal. In a preferred embodiment, the first monoclonal antibody is specific for complement receptor (CR1 or CD35) on a human erythrocyte and the isotype of the second monoclonal antibody is human IgG1 or human IgG3. In another embodiment, the first monoclonal antibody is specific for Factor H on a non-primate mammalian platelet and the isotype of the second monoclonal antibody is the isotype having the highest known affinity for the Fc receptor in said non-primate mammal.

Illustrative examples of cancers that can be treated according to the methods of the present invention include, but are not limited to, neoplasms, tumors, metastases, or any disease or disorder characterized by uncontrolled cell growth. Examples of types of cancer and proliferative disorders include, but are not limited to, leukemia (e.g., myeloblastic, promyelocytic, myelomonocytic, monocytic, erythroleukemia, chronic myelocytic (granulocytic) leukemia, and chronic lymphocytic leukemia), lymphoma (e.g., Hodgkin's disease and non-Hodgkin's disease), fibrosarcoma, myxosarcoma, liposarcoma, chondrosarcoma, osteogenic sarcoma, angiosarcoma, endotheliosarcoma, Ewing's tumor, colon carcinoma, pancreatic cancer, breast cancer, ovarian cancer, prostate cancer, squamous cell carcinoma, basal cell carcinoma, adenocarcinoma, renal cell carcinoma, hepatoma, Wilms' tumor, cervical cancer, uterine cancer, testicular tumor, lung carcinoma, small cell lung carcinoma, bladder carcinoma, epithelial carcinoma, glioma, astrocytoma, oligodendroglioma, melanoma, neuroblastoma, retinoblastoma, dysplasia and hyperplasia. In a particular embodiment, therapeutic compounds of the invention are administered to men with prostate cancer (e.g., prostatitis, benign prostatic hypertrophy, benign prostatic hyperplasia (BPH), prostatic paraganglioma, prostate adenocarcinoma, prostatic intraepithelial neoplasia, prostato-rectal fistulas, and atypical prostatic stromal lesions). The treatment and/or prevention of cancer includes, but is not limited to, alleviating one or more symptoms associated with cancer, the inhibition or reduction of the progression of cancer, the promotion of the regression of cancer, and/or the promotion of the immune response. The treatment and/or prevention of cancer also includes the clearance or reduction of metastatic cells circulating in the blood or lymph systems.

In certain embodiments, a heteropolymer complex of the invention is administered to a mammal to ameliorate one or more symptoms associated with cancer. In certain other embodiments, a heteropolymer complex of the invention is administered to a mammal to inhibit or reduce the progression of cancer. In certain other embodiments, a heteropolymer complex of the invention is administered to a mammal to promote the regression of cancer.

The heteropolymer complex or heteropolymer cocktail of the invention may be administered alone or in combination with other types of cancer treatments (e.g., radiation therapy, chemotherapy, hormonal therapy, immunotherapy and anti-tumor agents). Examples of anti-tumor agents include, but are not limited to, cisplatin, ifosfamide, paclitaxel, taxanes, topoisomerase I inhibitors (e.g., CPT-11, topotecan, 9-AC, and GG-211), gemcitabine, vinorelbine, oxaliplatin, 5-fluorouracil (5-FU), leucovorin, vinorelbine, temodal, and taxol. Such other types of cancer treatments may be administered to a mammal, preferably a human, for the prevention or treatment of cancer prior to (e.g., 1 minute, 15 minutes, 30 minutes, 45 minutes, 1 hour, 2 hours, 4 hours, 6 hours, 8 hours, 12 hours, 24 hours, 2 days, or 1 week before), subsequent to (e.g., 1 minute, 15 minutes, 30 minutes, 45 minutes, 1 hour, 2 hours, 4 hours, 6 hours, 8 hours, 12 hours, 24 hours, 2 days, or 1 week after), or concomitantly with the administration of the heteropolymer complex.

5.5 Demonstration of Therapeutic or Prophylactic Utility

Compositions of the present invention for use in prevention or therapy can be tested in suitable animal model systems prior to testing in humans, including but not limited to chimpanzees, monkeys, etc. For in vivo testing, prior to administration to humans, any appropriate animal model system known in the art may be used. See Reinagel and Taylor, 2000, J. Immunol. 164:1977, for an illustrative testing model.

5.6 Therapeutic/Prophylactic Administration and Composition

The invention provides methods of clearance of immune complexes, methods of preventing and treating viral infection or microbial infection, methods for preventing and treating septic shock, and methods for treating cancer by administering to a mammal (e.g., pigs, cats, dogs, rats, rabbits, guinea pigs, humans, etc.) an effective amount of a heteropolymer complex or heteropolymer complex cocktail composition of the invention. In certain embodiments, compositions of the invention are administered to human burn patients, infants (18 months old or less), immunocompromised or immunodeficient humans, or the elderly (greater than 60 years old). In a preferred aspect, a composition of the invention is substantially purified (e.g., substantially free from substances that limit its effect or produce undesired side-effects).

Various delivery systems are known and can be used to administer a composition of the invention, e.g., encapsulation in liposomes, microparticles, microcapsules, etc. Methods of introduction include, but are not limited to, intradermal, intramuscular, intraperitoneal, intravenous, subcutaneous, intranasal, epidural, and oral routes. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In another embodiment, the composition can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see, generally, ibid.).

In yet another embodiment, the composition can be delivered in a controlled release or sustained release system. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used in a controlled release system (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled release system can be placed in proximity of the therapeutic target (e.g., the brain, kidney, stomach, pancreas, and lung), thus requiring only a fraction of the systemic dose (see, e.g., Goodson, in Medical Applications of Controlled Release, supra, vol. 2, pp. 115-138 (1984)).

Other controlled release systems are discussed in the review by Langer (1990, Science 249:1527-1533).

The present invention also provides pharmaceutical compositions. Such compositions comprise a prophylactically or therapeutically effective amount of one or more heteropolymer complexes of the invention, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, pharmaceutical compositions for intravenous administration are solutions in sterile isotonic aqueous buffer. Where necessary, the pharmaceutical composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The pharmaceutical compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of a composition of the invention which will be effective in the treatment or prevention of viral infection or microbial infection can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

For heteropolymer complexes, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al., 1997, J. Acquired Immune Deficiency Syndromes and Human Retrovirology 14:193).

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### 6. EXAMPLE 1

The following example demonstrates that a bispecific heteropolymer complex, consisting of a monoclonal antibody (mAb) specific for the primate erythrocyte complement receptor cross-linked with an anti-bacterial mAb, targets bacteria in the bloodstream of monkeys in an acute infusion model. In vitro studies demonstrated a variable level of complement-mediated binding (immune adherence) of P. aeruginosa (strain PAO1) to primate erythrocytes in serum. In vivo experiments in animals depleted of complement revealed that binding of bacteria to erythrocytes was <1% before administration of the bispecific reagent, but within 5 minutes of its infusion, >99% of the bacteria bound to erythrocytes. In complement-replete monkeys a variable fraction of infused bacteria bound to erythrocytes. This finding may have significant implications in the interpretation of animal models and in the understanding of bacteremia in humans. Treatment of these complement-replete monkeys with the bispecific reagent led to >99% binding of bacteria to erythrocytes. Twenty-four hour survival studies were conducted; several clinical parameters, including the degree of lung damage, cytokine levels and liver enzymes in the circulation, indicate the bispecific mAb reagent provides a degree of protection against the bacterial challenge.

6.1 Introduction

Humans and other mammals have several lines of defense against bacteria and viruses which may invade the bloodstream (Brown et al., 1982, J. Clin. Invest. 69:85; Cross et al., 1993, Infect. Immun. 61:2741). Due to protection afforded by innate immunity, immunologically naive animals challenged intravenously with small to moderate doses of most bacteria can clear and destroy the bacteria by a variety of mechanisms which make use of pattern recognition receptors, natural IgM antibodies, and the complement system (Muller-Eberhard, 1989, Curr. Opin. Immunol. 2:3; Ulevitch et al., 1999, Curr. Opin. Immunol. 11:19; Ochsenbein et al., 1999, Science 286:2156). Binding of antibodies to antigens in immunized animals results in formation of immune complexes (IC) which can fix complement, capture the complement activation product C3b, and bind to immune adherence receptors on circulating cells (Nelson, 1953, Science 118:733; Cornacoff et al., 1983, J. Clin. Invest. 71:236; Taylor et al., 1997, J. Immunol. 159:4035; Fearon, 1980, J. Exp. Med. 152:20). More than 90% of these receptors (CR1) in primates are found in the circulation on erythrocytes (E). Nelson first demonstrated, both in vitro and ex vivo, that opsonization of bacteria with specific antibodies leads to complement-mediated binding of bacteria to primate E (Nelson, 1953, Science 118:733). His work and that of Robineaux suggests that immune adherence and immobilization of bacteria on primate E enhances their ingestion and destruction by phagocytic cells. This reaction may therefore be important in host defense against pathogens (Robineaux and Pinet. 1960, Ciba Found. Symp. Cell. Aspects Immun.: 5).

The goal of the following experiments is to evaluate the potential of the HP-E system to target bacteria in the bloodstream. An acute bacterial infection model is utilized, which model is based upon challenge with large doses of bacteria continuously infused i.v. over several hours (Brockmann et al., 1986, Am. Rev. Respir. Dis. 134:885; Creasey et al., 1991, Circ. Shock 33:84; Redl et al., 1996, Am. J. Physiol. 271:1193; Taylor et al., 2000, Blood 95:1680). Under these conditions live bacteria can be demonstrated in the circulation and a variety of effector mechanisms can be analyzed, although the role of immune adherence in this model has not, to our knowledge, been previously evaluated. The handling of Pseudomonas aeruginosa (strain PAO1) in the bloodstream of complement-depleted and complement-replete animals, with and without HP treatment, was studied. It was found that, although a variable fraction of bacteria infused into the circulation of complement-replete monkeys binds to E in the absence of HP, infusion of HP leads to >99% binding of bacteria to E. In addition, the results of experiments conducted with paired monkeys challenged with or without HP demonstrated that several parameters associated with resistance to the bacterial challenge are enhanced by HP treatment.

6.2 Materials and Methods

Monoclonal antibodies (MAbs). Anti-CR1 mAbs 7G9 and 9H3, specific for human and monkey E CR1, have been described (Craig et al., 1999, Clin. Immunol. 92:170; Ferguson et al., 1995, Arthritis Rheum. 38:190). MAbs specific for P. aeruginosa PAO1 (Holloway et al., 1994, Microbiol. 140:2907) and E. coli (strain 0 type 2, cytotoxic necrotizing factor type I isolated from the clinical microbiology laboratory, UVA Hospital) were generated from hybridomas after immunization of A/J mice with heat killed bacteria. Cell culture supernatants (CCS) produced by hybridomas were screened for specific mAbs by measuring binding to microtiter plates coated with bacteria. Selection for high avidity mAbs (Taylor et al., 1997, J. Immunol. 158:842) employed flow cytometry, RIA and magnetic separation. Bacteria were incubated with CCS, washed, and probed with FITC-labeled anti-mouse IgG, or ¹²⁵I-labeled anti-mouse IgG. Washed samples were analyzed by flow cytometry or monitored for bound ¹²⁵I, respectively. Alternatively, BioMag anti-mouse IgG coated iron particles (Polysciences Inc., Warrington, Pa.) were added to bacteria incubated in CCS. Free bacteria were separated from particle-bound bacteria in a Polysciences Magnetic Separation unit, and counted in a Coulter Multi-Sizer II (Coulter Co., Luton, England). MAbs which bound the bacteria in these assays were selected as high avidity mAbs. The anti-PAO1 mAb 2H4, isotype IgG2a, recognizes the LPS of PAO1 on Western blots (not shown) and the anti-E. coli mAb 3E1, is isotype IgG1.

Preparation of heteropolymers. mAbs were purified from ascites fluid or CCS by affinity chromatography (Chang et al., 1995, Meth. Enzymol. 254:430) and dialyzed exhaustively against borate saline (0.15 M NaCl, 0.03 M boric acid, pH 7.8). The cross-linking procedure was based on the method of Segal and Bast (Segal et al., 1995, Curr. Prot. Immunol. 2:13.1). The anti-CR1 mAb was reacted with N-succinimidyl S-acetylthioacetate (SATA, Pierce, Rockford, Ill.) at a ratio of 14 ug SATA/mg mAb for 2 h at room temperature (RT). The mixture was dialyzed with one change against HP buffer (50 mM sodium phosphate, 5 mM EDTA, pH 7.5) and then the SATA-mAb was deprotected to produce SH-mAb by treatment with 0.5 M hydroxylamine, 50 mM sodium phosphate, 25 mM EDTA, pH 7.5, under argon, for 2 h at RT. During this time, the anti-pathogen mAb was reacted with sulfosuccinimidyl 4-(N-maleimidomethyl)cyclohexane-1-carboxylate (sSMCC, Pierce) at a ratio of 14 ug sSMCC/mg mAb for 2 h at RT. At the end of these incubations, both the SH-mAb and the sSMCC-mAb were subjected separately to gel filtration in HP buffer on gravity flow 10DG columns (BioRad, Hercules, Calif.). Gel filtration of the SH-mAb was performed under a stream of argon, and the SH-mAb containing fractions were stored under argon for a minimum period of time until coupling. The SH-mAb and sSMCC-mAb were combined at a 10-20% (by weight) excess of SH-mAb, mixed gently by inversion, flushed with argon and reacted 16 h at RT with gentle shaking. The coupling reaction was stopped by incubation with 1 mg iodoacetamide/10 mg mAb for 1 h at RT, and then stored at 4° C. The coupling reaction mixture was subjected to gel filtration in borate saline buffer on Superose 6 (Pharmacia) which was calibrated with monomeric IgG mAb and human IgM. Crosslinked product eluting between, but fully excluding the positions of these two markers was pooled and used for experiments. Pooled product was stored at 4° C.

Construction of GFP-PA01 and GFP-E coli. Plasmid pSMC2 coding for green fluorescent protein (Bloemberg et al., 1997, App. Env. Micro. 63:4543) and β-lactam resistance was kindly provided by Dr. George O'Toole, Dartmouth Medical School, Hanover, N.H. The plasmid was transferred to P. aeruginosa PAO1 and the clinical isolate of E. coli by electroporation using standard procedures (Smith and Iglewski, 1989, Nuc. Acids Res. 17:10509; Provence and Curtiss, 1994, Gene transfer in Gram-negative bacteria. In Methods for general and molecular bacteriology. Gerhardt et al., Eds., ASM Press, p. 317.). Cultures were maintained on standard agar supplemented with 350 ug/ml carbenicillin and 100 ug/ml ampicillin, respectively.

Monkey anti-bacterial antibodies. The titers were determined by incubating GFP-PAO1 with varying dilutions of monkey plasma for 15 min at 37° C. Opsonized bacteria were washed three times, and probed with PE-labeled anti-monkey IgG or a PE-labeled mAb specific for human IgM which cross-reacts with monkey IgM. The IgG titers are reported as the reciprocal dilutions of plasma which caused 50% of the bacteria to register as FL2 positive by flow cytometric analysis. IgM titers (not shown) gave similar trends.

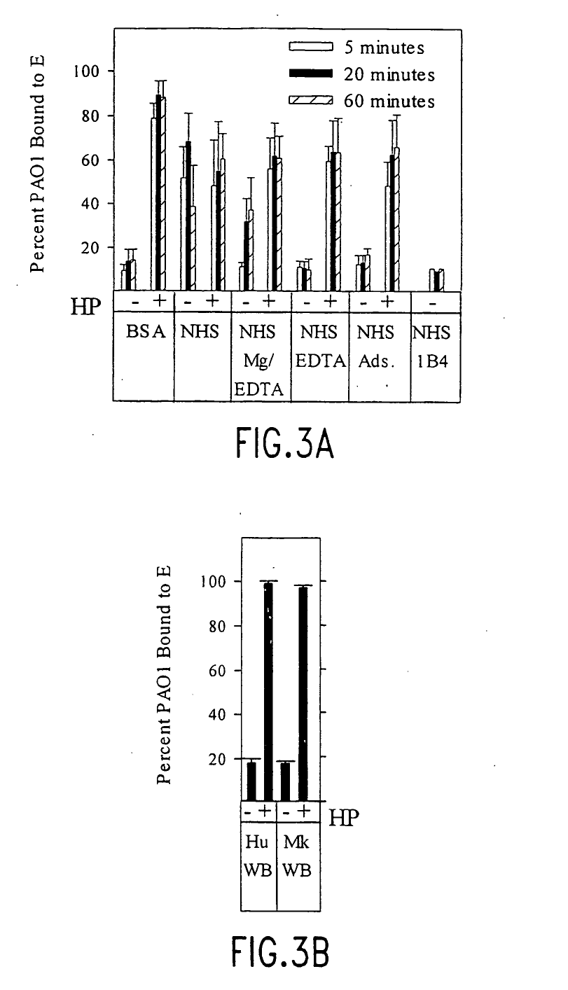

In vitro binding of GFP-PAO1 and GFP-E. coli to primate E. Measurement of HP- and/or serum-mediated binding of bacteria to E followed methods reported by Kuhn et al., 1998, J. Immunol. 160:5088. In brief, 10 ul (70 ng) of specific or irrelevant HP were added to 50 ul of a 50% E dispersion in either 1% BSA in PBS (BSA-PBS) or in a blood group matched serum. After 5 min at 37° C., 5×10⁶ GFP-transformed bacteria were added, giving an E/bacterium ratio of ˜50 to 1 (FIG. 3A). Mixtures were incubated for 5 to 60 min at 37° C., and an aliquot was diluted into iced BSA-PBS. Samples were analyzed by flow cytometry (Becton Dickinson FacsCalibur) by gating for FL1 positive events. Percent of bacteria either bound to E or free was determined by examination of the forward and side scattering profiles of FL1 events for the two populations. In some assays HP were added to mixtures of whole blood and bacteria at much higher E to bacterium ratios (>500 to 1), and after incubation and centrifugation (100×g, 5 min), the number of bacteria free in the supernatant was determined by flow cytometry and compared to matched samples in which either an irrelevant HP or no HP was added (FIG. 3B). Equal volumes of the samples were examined in each case. No evidence was found for serum-mediated killing of GFP-PAO1 for an incubation period of 1 h at 37° C. based on CFU assays (not shown).

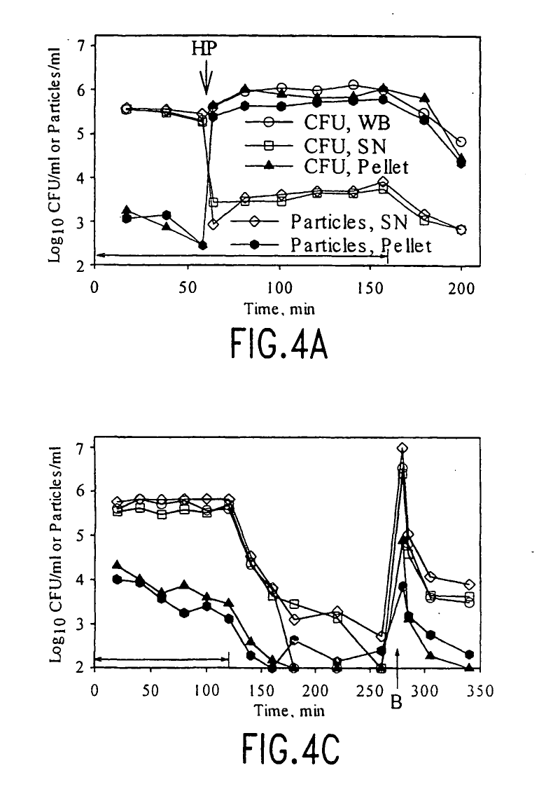

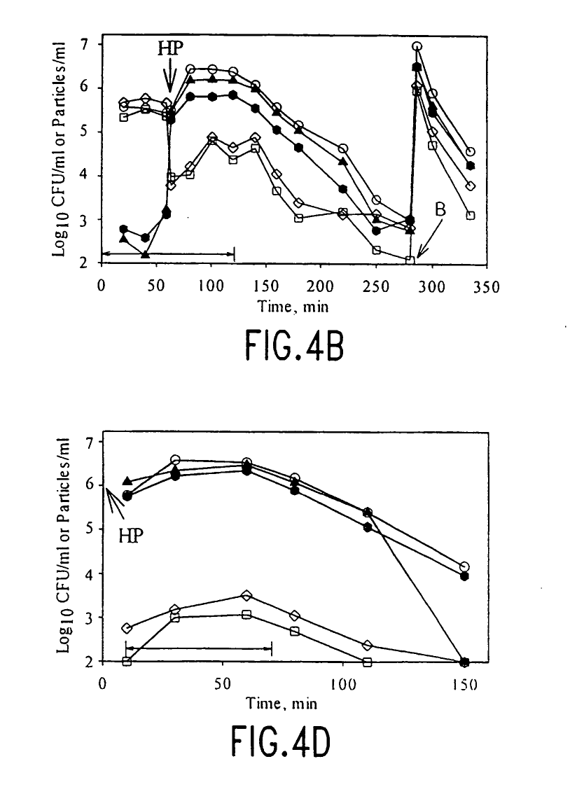

In vivo protocol. All animal experiments were supervised by a qualified veterinarian in accordance with approved protocols of the University of Virginia Animal Care and Use Committee and the Institutional Biosafety Committee. Cynomolgus and rhesus monkeys weighed between 2.3 and 8.5 kg. RIA (Craig et al., 1999, Clin. Immunol. 92:170; Ferguson et al., 1995, Arthritis Rheum. 38:190) conducted with anti-CR1 mAbs demonstrated 1000-3000 CR1 epitopes per E, with the exception of one monkey (see FIG. 4C). Some monkeys were pre-treated i.v. with cobra venom factor (CVF, 70 units/kg, Quidel, San Diego, Calif.) 24 h before GFP-PAO1 infusion to consume complement (Taniguchi et al., 1996, Transplant 62:678). Hemolytic complement activity (CH50) determinations revealed little to no residual complement activity 24 h later. Prior to the bacterial infusion, the monkey was anesthetized (ketamine 10 mg/kg i.m., atropine 0.04 mg/kg s.c.), intubated, and maintained under anesthesia with isoflurane and 100% oxygen. Blood pressure was monitored through a catheterized femoral artery (MicroMed Inc., Louisville, Ky.).

An i.v. infusion of Lactated Ringers Solution was established during the first hour of the experiment at a rate of 10 ml/kg/h. Hypotension is a common hemodynamic effect associated with isoflurane anesthesia in macaques (Popilskis and Kohn, 1997, Anesthesia and analgesia in nonhuman primates. In Anesthesia and analgesia in laboratory animals Kohn et al., Eds. Academic Press, New York, p. 233), and was also seen as a direct response to the bacterial infusion. When indicated, phenylephrine was infused in boluses or at a calculated dosage of 0.5-3 ug/kg/min, with the goal of maintaining mean blood pressure above 50 mm Hg.

An overnight agar culture of GFP-PAO1 was suspended in PBS, washed three times, suspended in sterile saline (at ˜1×10⁹ CFU/ml) and infused into the cephalic vein over a period of 1-4 h (see FIGS. 4-6) at infusion rates corresponding to ˜10⁹ CFU/kg/h. Blood samples were drawn through an arterial catheter, and HP preparations or mAbs were infused as a bolus, 3-5 ml over 60 seconds, through the opposite cephalic vein. At the end of the experiment all animals were humanely euthanized under anesthesia. Samples of liver, lungs and spleen were fixed in 10% formalin and submitted, blinded, for examination by a pathologist. In addition, in selected experiments samples of organs were homogenized as 20% dispersions in sterile filtered 0.1% Triton in PBS, and diluted aliquots of these dispersions were analyzed for CFU.

Processing of blood samples. Blood samples were anti-coagulated with EDTA, held on ice and processed within 15 min. Plasma supernatants were taken after centrifugation (100×g, 5 min). The pellet was washed once at 200×g and twice at 1800×g, and the buffy coat was removed during washing. A 10 ul aliquot of this washed E pellet was lysed by dilution into 0.5 ml of distilled water followed by vigorous vortexing, and 0.5 ml of 2×PBS was added. Both the plasma and lysed E pellet were analyzed for GFP-PAO1 by flow cytometry based on a series of in vitro calibrations. The method for analysis of the E pellet is based on the controlled acquisition of an identical volume (500 ul out of 1 ml) of each sample at constant flow. An FL1 threshold was set to record fluorescent events, and then a forward/side scattering window of sufficient size was selected to include all bacteria in the cell lysate, including those that may have been associated with E membrane fragments. In all experiments the E were in far excess over bacteria, and so the likelihood of undercounting of bacteria due to their coincident binding to the same E was minimized. The number of E in the lysed preparation was determined by measurement of the absorbance at 541 nm of the residual uncounted sample. A hematocrit (HCT) of 0.4% corresponds to an absorbance of 0.81 after lysis. Based on these determinations and the HCT, we calculated the concentration of GFP-PAO1 bound to E (designated Particles, Pellet in FIGS. 4-6) and free in the plasma (designated Particles, SN). The plasma supernatants were diluted into sterile-filtered BSA-PBS and measured volumes of 0.5 ml were counted for fluorescent bacteria using the same FL1 cutoff and a similar light scattering gate. Replicate samples of whole blood, plasma supernatant and pelleted E were analyzed for CFU (designated CFU, Whole Blood; CFU, SN; CFU, Pellet; respectively, in FIGS. 4-6). When levels were so low that bacteria could not be detected by CFU assay in the most concentrated samples, the value is reported as 100 CFU/ml in FIGS. 4-6.

Separate aliquots of blood were washed three times, the buffy coat was removed, and isolated E were reconstituted in BSA-PBS and probed with either an ¹²⁵I-labeled anti-CR1 mAb (the same as used to prepare the HP) or with ¹²⁵I-labeled goat anti-mouse IgG. Approximately 10⁸ E were incubated with 0.2-1 ug of ¹²⁵I-labeled probe for 30 min at 37° C., and after three washes or centrifugation through oil (Craig et al., 1999, Clin. Immunol. 92: 170; Ferguson et al., 1995, Arthritis Rheum. 38:190) the amount of ¹²⁵I bound to the E was determined. The E concentration in these samples was determined as described above. Additional aliquots of blood were centrifuged at 3000×g to generate plasma supernatants which were stored at −80° C. for cytokine determinations. A total of 10-15% of the animals' blood volumes were taken, and as much as 150 ml Lactated Ringers Solution was infused; the HCT of both the control and HP-treated monkeys showed comparable decreases.

Cytokine assays. An ELISA sandwich assay was used to measure cytokines (TNF-α, IL-1β, IL-6) in the plasma of monkeys. Plates were coated with the appropriate anti-cytokine capture mAb (Pharmigen, San Diego, Calif.), incubated with diluted plasma and then with a biotinylated mAb which did not compete with the capture in Ab. Development was accomplished by addition of neutralite avidin coupled to horseradish peroxidase (Southern Biotechnology, Birmingham, Ala.). Standards included recombinant rhesus monkey TNF-α (Biosource International, Camarillo, Calif.), human IL-1β and human IL-6 (Pharmigen, San Diego, Calif.).

6.3 Results

In vitro assays. HP specific for binding of GFP-PAO1 to CR1 on primate E were tested in vitro with human and monkey E, in preparation for in vivo studies in monkey models. FIG. 3A shows the degree of GFP-PAO1 binding to human E under a variety of conditions, at a ratio of approximately 50 E/bacterium. In BSA-PBS less than 15% of GFP-PAO1 bacteria are bound to E. Addition of specific HP promoted >90% binding of GFP-PAO1 to E in BSA-PBS, and this binding is rapidly attained and stable over 60 min. In the presence of serum (no HP added), where complement activation should lead to deposition of C3b on GFP-PAO1, E binding reaches 68% in 20 min, but then binding decreases, presumably as C3b decays to C3bi and C3dg. It is likely that E binding is mediated by activation of the classical pathway of complement because binding is lower during the first 20 min in serum containing Mg-EGTA which only allows for alternative pathway activation and binding is abrogated if serum is treated with EDTA (FIG. 3A). Pre-adsorption of serum with bacteria on ice greatly reduces the ability of serum to facilitate GFP-PAO1-E binding, suggesting that the sera contain complement-fixing antibodies specific for the bacteria. MAb 1B4, which blocks the C3b binding site on human E (O'Shea et al., 1985, J. Immunol. 134:2580; Edberg et al., 1987, J. Immunol. 139:3739), inhibits binding as does heat inactivation of serum (not shown). We find that in whole serum it is difficult to demonstrate HP-mediated binding (FIG. 3A), because the natural process of complement-mediated immune adherence leads to a high level of binding in the absence of HP. However, as noted above, serum-mediated E binding decreases by 60 min, and at this time point the differences between the HP-treated and control samples in serum achieve modest statistical significance, 61±11% versus 39±19%, p=0.042, unpaired t-test (FIG. 3). If the bacteria are suspended in BSA-PBS, in serum EDTA, or in adsorbed sera, then HP-mediated binding is demonstrable (FIG. 3A). However, the level of binding in BSA-PBS (>90%) was always higher than in samples which contained plasma, even if complement was inhibited.

HP-mediated binding of GFP-PAO1 to E was next tested in whole blood anti-coagulated with EDTA, and in order to more closely simulate physiological conditions expected in the bloodstream, where E would be in great excess over bacteria (Shenep et al., 1988, J. Infect. Dis. 157:565; Kreger et al., 1980, Amer. J. Med. 68:332), a ratio of 500E/bacterium was used. The results (FIG. 3B) demonstrate HP-mediated binding of GFP-PAO1 to both monkey and human E; at least two log units of bacteria are bound to E in these experiments. Finally, under similar experimental conditions, both serum and specific HP mediate substantial binding of another Gram-negative bacterium, E. coli, to primate E. In BSA-PBS HP-mediated binding of GFP-E. coli to human and monkey E averaged >90%; binding in serum (no HP added) averaged 80 and 95% for human and monkey E respectively, based on 3-6 independent determinations.

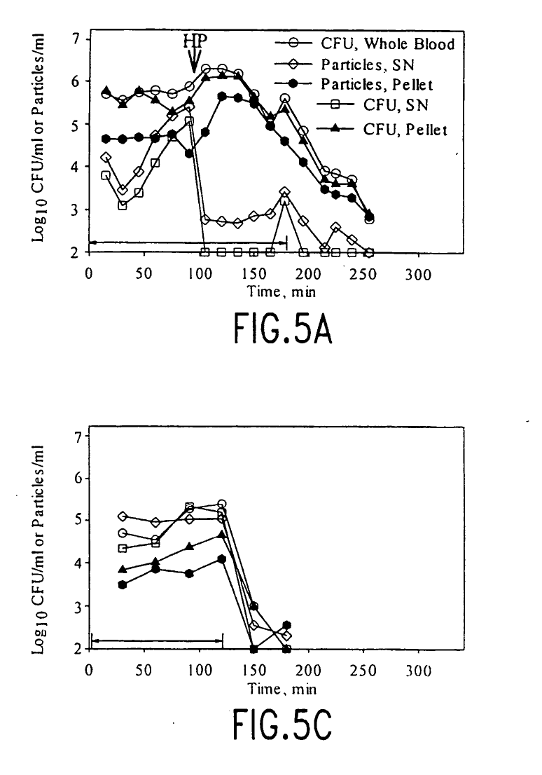

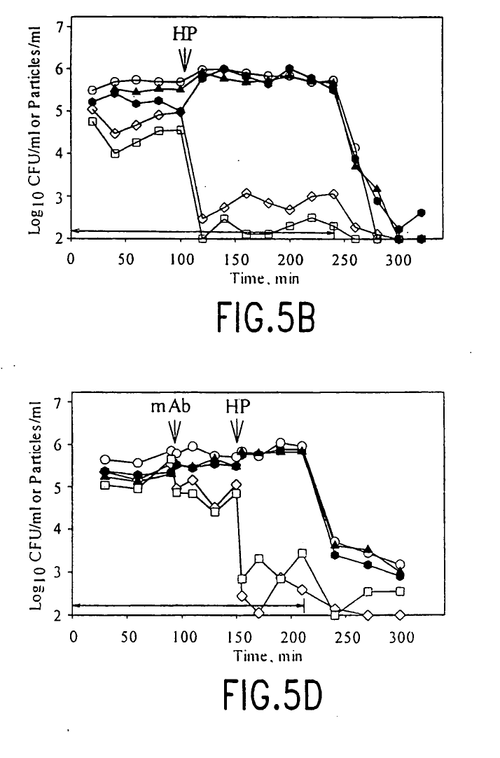

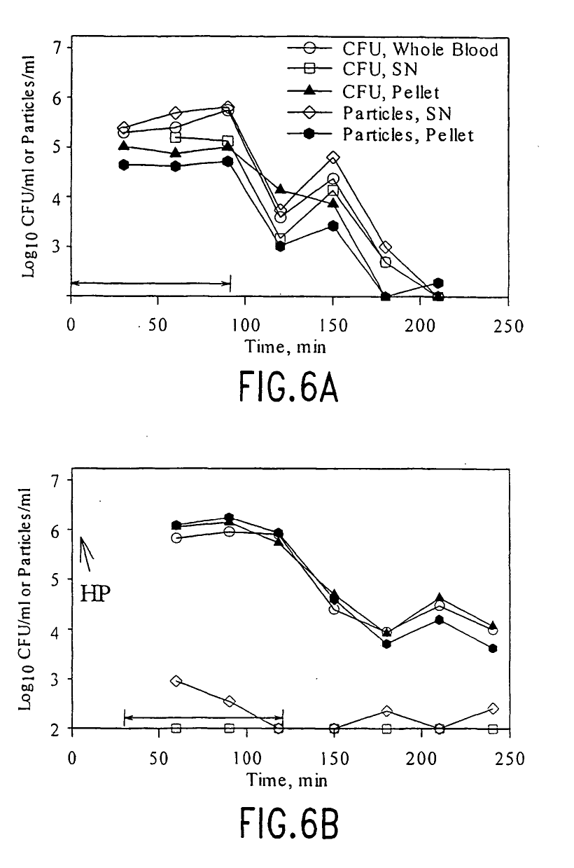

Complement-depleted monkeys: in vivo HP-mediated binding of GFP-PAO1 to E. In view of the natural, physiologic effects of complement in facilitating binding of bacteria to E in the primate system, HP in a monkey model in which animals were pre-treated with CVF to consume complement was tested. 24 hours after CVF treatment, bacteria was infused, in the expectation that by this time most complement activation products would be cleared from the bloodstream, and complement receptors, especially E CR1, would be available for ligation. As shown in FIG. 4A, continuous infusion of GFP-PAO1 led to negligible binding of bacteria to E over the first hour of the experiment. When a bolus of HP was infused the number of GFP-PAO1 that circulated freely in the plasma decreased by ˜100 fold, the number of bacteria bound to E increased by a factor of 500 and the total number of bacteria in the bloodstream increased. The initial effect of the HP was observed within a few minutes of infusion, and persisted for the remainder of the experiment. After the bacterial infusion was stopped at 160 min, the levels of both E-bound and free bacteria decreased. GFP-PAO1 bound to E and free in plasma was analyzed by flow cytometry and by determination of CFU (see Materials and Methods, supra). In this experiment and those described below (FIGS. 5, 6, Table I) there was generally good agreement between the flow cytometry measurements (Particles) and the CFU assays with respect to the number of bacteria determined as either bound to E or as free in the plasma.

In the next experiment (FIG. 4B) the same dose of bacteria was infused over a shorter time period. No binding of GFP-PAO1 to E in the CVF-treated animal before administration of HP was observed; after HP infusion >99% of the bacteria were bound to E and the total number of bacteria in the circulation increased. After the GFP-PAO1 infusion was terminated at 120 min, levels of GFP-PAO1 decreased substantially. A second bolus of GFP-PAO1 was infused at 260 min, and >70% of the GFP-PAO1 immediately bound to E and >90% of the GFP-PAO1 was removed from the circulation after 60 min. The results from a control experiment (CVF treatment, but no HP), shown in FIG. 4C, indicate a more rapid disappearance of bacteria from the circulation after the GFP-PAO1 infusion was stopped at 120 min. In the absence of HP, there was little binding of GFP-PAO1 to E throughout the experiment for this control monkey. The steady state level of bacteria in the circulation was lower than that observed in FIG. 4B after HP infusion, suggesting that bacteria free in the bloodstream (not bound to E) leave the circulation more rapidly.

Finally, it was found that pretreatment of a complement-depleted monkey with HP before infusion of bacteria led to a very high level of E-associated binding when bacteria were infused (FIG. 4D). Fluorescence microscopy confirmed that in the presence of HP the bacteria were bound to E; however the vast majority of the E had no bound bacteria (not shown), as expected for ˜10⁷ bacteria/ml versus 4×10⁹ E/ml.

Complement-replete monkeys: in vivo HP-mediated binding of GFP-PAO1 to E. It was investigated next whether HP could be used to unambiguously bind GFP-PAO1 in complement-replete monkeys (FIGS. 5A, 5B), which represent a more physiologically relevant condition. It was found that continuous infusion of bacteria into either cynomolgus or rhesus monkeys leads to a steady state in which a variable fraction of the bacteria in the circulation are bound to E. This observation, coupled with the absence of immune adherence in CVF treated animals, argues that complement activation must play a role in binding GFP-PAO1 to E. When HP was infused, the substantial changes in the distribution of E-bound and free bacteria observed in the CVF-treated monkeys were again demonstrable (FIGS. 5A, 5B). The number of bacteria free in the circulation decreased precipitously while the number bound to E increased and the total number of bacteria in the bloodstream increased 2-4 fold. After HP infusion, >99.9% of bacteria in the bloodstream were associated with E. These rapid changes in the distribution of E-bound and free bacteria were not observed in a control monkey where HP was not infused (FIG. 5C).

Both the in vitro and in vivo results (FIGS. 3, 5) suggest that, in the absence of HP, the binding of the bacteria to primate E is facilitated to a great extent by anti-PAO1 antibodies which promote complement activation (Muller-Eberhard, 1989, Curr. Opin. Immunol. 2:3; Ochsenbein et al., 1999, Science 286:2156; Nardin et al., 1999, Mol. Immunol. 36:827). The isotype of anti-PAO1 mouse mAb 2H4 is Ig2a, which is capable of fixing complement, and therefore it could be argued that the enhanced HP-mediated binding of GFP-PAO1 to E in the bloodstream of the monkeys might be due to complement activation after mAb 2H4 binds to the bacteria. In order to examine this possibility, mAb 2H4 was infused into the circulation of a cynomolgus monkey during a continuous infusion of GFP-PAO1 (FIG. 5D). Before mAb treatment, E binding was ˜50%. Infusion of the mAb alone led to an increase in E-bound PAO1 and to a decrease in PAO1 in plasma, consistent with enhanced immune adherence (FIG. 5D, Table I). However, when an equimolar amount of HP was later infused, the number of bacteria free in the plasma decreased substantially, the total number of bacteria in the bloodstream increased two-fold, and >99% of the bacteria in the bloodstream were bound to E (FIG. 5D, Table I). This result is in agreement with our previous findings which indicate that at equivalent doses, mAbs alone are not as effective at promoting in vivo binding of the target pathogens to primate E, compared to the same mAbs when they are formulated into the HP (Taylor et al., 1997, J. Immunol. 158:842).

There was no evidence that HP infusion caused E destruction. Total bilirubin levels remained low (<0.3 mg/dL, not shown) before and after HP infusion for all monkeys. The decreased HCT at the end of some experiments (see brief description of the figures) are expected after withdrawal of 10-15% of the total blood volume and infusion of fluids. We have previously demonstrated negligible loss of autologous E when ⁵¹Cr-labeled E were opsonized with ¹²⁵I-labeled substrates (both proteins and E. coli) via HP and infused into a monkey (Nardin et al., 1999, Mol. Immunol. 36:827).

Complement-replete monkeys: treatment with HP before infusion of bacteria. It was next investigated how pre-treatment of monkeys with HP would affect the animals' short-term responses to bacterial challenge. The experiment was designed to examine several clinical parameters, in particular lung damage, over a 24 h time period after GFP-PAO1 infusion in the presence of HP but in the absence of antibiotics. A naive animal was compared to a HP-treated monkey for three different infusion doses of bacteria. The results indicate that for each GFP-PAO1 dose, more bacteria were free in the plasma in the naive animals compared to the HP-treated animals (Table IIA, FIG. 6). However, binding of PAO1 to E was clearly evident in the untreated monkeys. The levels of immune adherence roughly correlated with the titers of monkey IgG antibodies for PAO1. For example, monkey 6A (FIG. 6) had only moderate binding (32%, Table IIA) and had a reciprocal titer of 5. More than 60% of the infused bacteria bound to the E of monkeys 5B (before HP infusion), 7A, and 7C (FIG. 5, Table IIA), and these monkeys had reciprocal titers of IgG for PAO1 of 40, 50, and >100, respectively.

The clinical condition of the HP-treated monkeys was better than that of the naive animals, based on subjective criteria and clinical analyses. For example, elevation of liver enzymes in the HP-treated animals was lower than in the untreated animals (Table IIA). Organs were analyzed for CFU's (Table IIA), and although no statistically significant conclusions can be drawn from such a small number of animals, the trend is toward lower levels of viable bacteria in the organs of the HP-treated animals. Table IIB summarizes the findings from the necropsy/pathology reports. Particularly striking was the level of protection from lung damage of the HP-treated animals at the higher doses of bacteria. Although there was no evidence for bacterial growth in the lungs of the control monkey (7A) treated with 3×10⁹ CFU/kg (Table IIA), postmortem evaluation of the lungs revealed congestion, fluid in the airways and histopathologic confirmation of the gross observations. At the highest dose of 6×10⁹ CFU/kg, there was a much greater difference between the naive and HP-treated monkey (7C and 7D, respectively) at the 24 h point. While only two very small foci of infection were detected on the lungs of the HP-treated monkey (7D, Table IIB), severe pathology was evident in the lungs of the control animal (7C), which also presented with infection of the lungs, heart and kidneys (Table IIA).

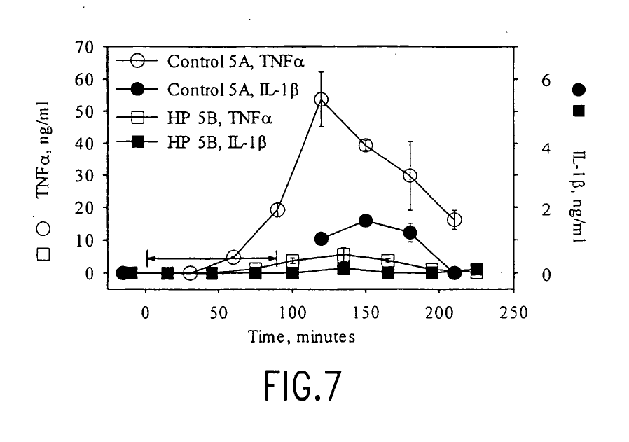

Reduction of Inflammatory Cytokine Levels by HP. Septic shock, one of the most important consequences of infection by Gram-negative bacteria, is mediated by LPS (Warren, 1997, N. Engl. J. Med. 336:952; Deitch, 1998, Shock 9:1; Morrison et al., 1999, Infect. Dis. Clin. No. Amer. 13:313). HP-mediated binding of GFP-PAO1 to E reduced substantially the level of free bacteria in the bloodstream, and it seemed reasonable that redirection to a clearance pathway which includes E binding might also affect the inflammatory potential of bacterial LPS. The mechanisms by which LPS interacts with plasma proteins and cell-surface receptors to initiate inflammation are complex. However, it is well-established that one of the earliest events in the inflammatory pathway is the appearance in the bloodstream of inflammatory cytokines such as TNF-α, IL-1β, and IL-6 (Redl et al., 1996, Am. J. Physiol. 271:1193; Morrison et al., 1999, Infect. Dis. Clin. No. Amer. 13:313; Hesse et al., 1988, Surg. Gynecol. Obstet. 166:147; Jansen et al., 1996, J. Immunol. 156:4401). Several groups have used primate models to delineate the kinetic profile of cytokine appearance in the circulation upon challenge with E. coli (Creasey et al., 1991, Shock 33:84; Redl et al., 1996, Am. J. Physiol. 271:1193; Hesse et al., 1988, Surg. Gynecol. Obstet. 166:147; Jansen et al., 1996, J. Immunol. 156:4401). The observations herein show that the cytokine release pattern after challenge with GFP-PAO1 is quite similar (Table III, FIG. 7). TNF-α levels increase in the circulation ˜1 h after the bacterial infusion is initiated, peak after 90-120 min and decrease thereafter. The increase in TNF-α is followed by an increase in levels of IL-1β and IL-6. These results indicate that use of HP dampens significantly the increase in cytokines promoted by the bacterial infusion. This finding strongly suggests that HP facilitate redirection and clearance of the bacteria by a pathway which may substantially reduce inflammation (see Discussion, infra).

Some of the HP and control animals were treated with phenylephrine (Table III, FIG. 7) to control blood pressure. It is unlikely that this treatment is responsible for the reduced cytokine levels in the HP-treated animals because phenylephrine is an α-adrenergic agonist and does not block TNF-α production (Severn et al., 1992, J. Immunol. 148:3441; Van Der Poll et al., 1996, J. Clin. Invest. 97:713; Van Der Poll et al., 1997, J. Exp. Med. 185:1143). Moreover, in the animals not treated with phenylephrine the same trends are evident.

E-HP and E CR1 levels. Isolated and washed E were examined by RIA for bound HP and relative CR1 levels. The results indicate that the HP rapidly binds to E, because infusion of HP led to a substantial increase in the amount of E-bound ¹²⁵I-labeled anti-mouse IgG (Table IV). As the experiment progressed the amount of this probe which could bind to the E decreased, suggesting that HP were being removed from the E, as we have previously demonstrated in similar systems (Reist et al., 1994, Eur. J. Immunol. 24:2018; Craig et al., 1999, Clin. Immunol. 92:170). E probed with anti-CR1 mAb 7G9 used to prepare the HP evidenced only small decreases in mAb binding immediately after HP infusion, which would be expected since the HP infused into the monkeys was sufficient to occupy ˜30% of total CR1, and some re-equilibration between free mAb and E-bound HP might have occurred during the in vitro incubations. It is noteworthy, however, that at later time points the amount of anti-CR1 probe that bound to the E further decreased, and these results follow the same patterns we have reported previously, in which clearance of E-bound HP occurs concomitantly with loss of E CR1 (Reist et al., 1994, Eur. J. Immunol. 24:2018; Nardin et al., 1999, Mol. Immunol. 36:827; Craig et al., 1999, Clin. Immunol. 92:170).

6.4 Discussion

In vivo evidence for HP-mediated binding. The goal of this study was to determine the ability of the HP system to target GFP-PAO1 in the bloodstream and to bind the bacteria to E during an i.v. challenge. HP were able to facilitate a very high level of binding of GFP-PAO1 to human and monkey E in BSA-PBS (FIG. 3A) and in anti-coagulated whole blood at higher E/PAO1 ratios (FIG. 3B). At lower E/PAO1 ratios (FIG. 3A), in the presence of NHS, there was a variable level of immune adherence, and HP-mediated binding to E could not easily be distinguished from natural complement-mediated binding. When complement activation was blocked, HP clearly promoted E binding, but the presence of the plasma proteins may have reduced HP-mediated binding in vitro. However, experiments in both complement-depleted and complement-replete monkeys (FIGS. 4-6) clearly demonstrate the very high level of efficiency by which HP promotes binding of the bacteria to E in vivo.

When the anti-PAO1 mAb 2H4 was used alone, an increase in complement-mediated immune adherence of bacteria to E was observed (FIG. 5D, 91 min, Table I). However, subsequent use of the HP containing this mAb at equal concentrations was far more effective in promoting E binding (FIG. 5D, 151 min, Table I). It is likely that this enhanced E binding mediated by the HP occurs because the anti-CR1 mAb in the HP, which acts as a surrogate for C3b (Nardin et al., 1999, Mol. Immunol. 36:827), binds to CR1 with a higher avidity than C3b, and therefore substantially increases ligation to CR1. It has been found that a concentration of 0.13 ug/ml of anti-CR1 mAb 7G9 is sufficient to achieve 50% saturation of E CR1, which corresponds to an association constant in excess of 10⁹ M⁻¹ (Lindorfer et al., 2001, J. Immunol. Methods 248:125), whereas the avidity of monomeric C3b for CR1 is >100 fold less (Arnaout et al., 1981, J. Immunol. 127:1348; Ahearn et al., 1989, Adv. Immunol. 46:183). Therefore, successful immune adherence requires that multiple C3b molecules deposit on a substrate and engage clusters of CR1 on the E to assure multivalent binding (Edberg et al., 1987, J. Immunol. 139:3739). The present work, including the findings in both the CVF-treated and complement replete monkeys, confirms earlier studies which indicate that the HP construct functions quite effectively in promoting in vivo binding of substrates to E CR1 (Hahn et al., 2001, J. Immunol. 166:1057; Reist et al., 1994, Eur. J. Immunol. 24:2018; Taylor et al., 1997, J. Immunol. 158:842).

mAb 2H4 was used alone in the in vivo immune adherence test (FIG. 5D) rather than an irrelevant HP (i.e., 2H4×IgG) because the HP themselves do not activate complement when bound to a variety of substrates. For example, of relevance to the present work, flow cytometry experiments indicated that incubation of the 7G9×2H4 HP with E and NHS in solution, or incubation of preformed E-HP complexes with NHS, gave negligible deposition of C3b on the E (not shown). These observations are in agreement with the work of Meri and colleagues (Jokiranta and Meri, 1993, J. Immunol. 151:2124; Hakulinen and Meri, 1998, Am. J. Pathol. 153:845). Synthesis of the HP makes use of N-hydroxysuccinimide chemistry to derivatize lysines on the mAbs (see Materials and Methods), and Jokiranta and Meri have reported that such chemical modification of mAbs blocks classical complement activation by blocking binding of C1q (Jokiranta and Meri, 1993, J. Immunol. 151:2124).

Immune adherence. Most animals appear to develop antibodies against common bacteria (Ochsenbein et al., 1999, Science 286:2156; Carroll, 1998, Ann. Rev. Immunol. 16:545; Parker et al., 1994, J. Immunol. 153:3791) and therefore it is not surprising that our in vitro and in vivo experiments demonstrate binding of GFP-PAO1 and E. coli to both monkey and human E under conditions allowing for activation of complement (FIGS. 3, 5, 6, Table I). Although the acute bacterial infusion model has been extensively tested and described in a variety of non-human primates (Brockmann et al., 1986, Am. Rev. Respir. Dis. 134:885; Creasey et al., 1991, Circ. Shock 33:84; Redl et al., 1996, Am. J. Physiol. 271:1193; Taylor et al., 2000, Blood 95:1680; Hesse et al., 1988, Surg. Gynecol. Obstet. 166:147; Jansen et al., 1996, J. Immunol. 156:4401), to our knowledge there have been no attempts to determine whether bacteria were bound to E or free in the plasma. In addition, although there is an extensive literature describing human clinical conditions associated with bacteremias, these reports have not revealed whether the bacteria in the bloodstream were free in the plasma or bound to E (Shenep et al., 1988, J. Infect. Dis. 157:565; Kreger et al., 1980, Amer. J. Med. 68:332; Weinstein et al., 1997, Clin. Infect. Dis. 24:584). Our results demonstrate immune adherence of bacteria to E in the non-human primate infusion model. As the degree of immune adherence of bacteria to E in the circulation is undoubtedly related to several factors including the levels of complement and anti-bacterial antibodies, it is possible that quantitative measurements of immune adherence may provide important prognostic information for patients with bacteremia. Similarly, it would seem important to determine whether bacteria infused into the bloodstream of mice or rabbits are bound to platelets, which contain the non-primate immune adherence receptor (Taylor et al., 1985, J. Immunol. 134:2550; Edberg et al., 1989, Clin. Immunol. Immunopath. 51:118).

Effects of HP on Bacterial Clearance. Experiments which compared HP-treated and naive monkeys indicate that HP-mediated binding of GFP-PAO1 to E tends to maintain the bacteria in the circulation for longer periods (compare 4B versus 4C, FIG. 4, and 6B versus 6A, FIG. 6). That is, the rate of removal of GFP-PAO1 from the vasculature was faster in the untreated monkeys, since both the steady state levels of bacteria in the circulation were lower, and the bacteria left the bloodstream more rapidly after the bacterial infusion was stopped. In contrast, after a monkey was treated with HP, the new steady state level of bacteria in the circulation increased and, when the infusion ended, bacteria bound to E were removed from the circulation at a slower rate. An important question focuses on the fate and organ distribution of the bacteria after they exit the bloodstream in the untreated versus the HP-treated monkeys. The slower rate of clearance of GFP-PAO1 bound to E via HP may reflect a different clearance mechanism, due to a rate determining step which requires scission of CR1 (Reist et al., 1994, Eur. J. Immunol. 24:2018; Nardin et al., 1999, Mol. Immunol. 36:827; Reinagel et al., 2000, J. Immunol. 164:1977) by proteases associated with fixed tissue macrophages in the liver and spleen, followed by uptake of the bacteria in these organs. Clearance through this mechanism should decrease the rate at which otherwise free (not E-bound) bacteria can invade other organs and tissues including the lungs, which are particularly susceptible to PAO1 (Kurahashi et al., 1999, J. Clin. Invest. 104:743; Engel et al., 1998, J. Biol. Chem. 273:16792). The decreased pathology associated with the lungs in the HP-treated monkeys (see above) is consistent with this hypothesis. Therefore, it is reasonable to anticipate that upon HP treatment a larger fraction of the bacteria will be redirected to the liver and spleen where the bacteria will be phagocytosed and destroyed. We measured live bacteria associated with these organs (Table IIA); if the bacteria were indeed killed they would not register in the CFU assay. There was no evidence for increased liver pathology as a result of HP treatment, and in fact the levels of liver enzymes in the circulation tended to be lower in HP-treated animals (Table IIA). Finally, comparison of FIGS. 6A and 6B suggests that in the control monkey the bacteria that bound to E by immune adherence are cleared faster than bacteria bound to E via HP in the treated monkey. It is likely that, with respect to binding of PAO1 to E, more HP (compared to C3b) engage a greater number of CR1 with higher avidity. It is therefore reasonable to expect that clearance of HP-bound bacteria would be slower, presumably because more CR1 molecules would have to be cleaved to allow transfer of the HP-bacteria complex to acceptor macrophages.

Effect of HP on Cytokine Release. Recognition of bacterial-associated structures such as LPS by plasma proteins and cellular receptors such as CD14/Tlr4 constitutes an important element in defense against bacterial invasion. High levels of LPS which are processed via the CD14 pathway can, however, provoke an exaggerated inflammatory response, generally signaled by an increase in cytokines in the circulation, which is ultimately damaging to the host (Warren, 1997, N. Engl. J. Med. 336:952; Deitch, 1998, Shock 9:1; Cross et al., 1995, J. Clin. Invest. 96:676; Poltorak et al., 1998, Science 282:2085). In monkeys treated with HP, >99% of bacteria in the bloodstream were bound to E, and the reduced cytokine levels in these animals suggests that processing of bacteria in these animals may have been different from processing of bacteria in the untreated animals. It is likely that bacteria bound to E via HP are more efficiently phagocytosed and destroyed by fixed tissue macrophages via a pathway that presumably utilizes Fc receptors on the macrophages (Reinagel and Taylor, 2000, J. Immunol. 164:1977; Heumann et al., 1992, J. Immunol. 148:3505; Pollack et al., 1997, J. Immunol. 159:3519; Aderem and Underhill, 1999, Ann. Rev. Immunol. 17:593). Under these conditions the bacteria and LPS in particular could therefore be redirected away from pathways which engage the CD14 receptor and might otherwise provoke an inflammatory response mediated by cytokines such as TNF-α. We recognize that the number of monkeys used in the present study is limited, and that statistically significant comparisons can not be made based on such a small sample size.

In summary, we have examined how treatment with HP affects handling of GFP-PAO1 in the bloodstream of monkeys. A fraction of infused bacteria bind to E via immune adherence, a complement-mediated reaction. However, infusion of a HP specific for GFP-PAO1 and E CR1 leads to a much higher level of binding of the bacteria to E (>99%), and to a substantially reduced level of bacteria free in the plasma. Based on the results presented, it is concluded that E-bound bacteria have less opportunity to colonize susceptible organs, and in addition are cleared from the circulation by a mechanism which bypasses to a great extent the CD14/LPS inflammatory pathway. Several clinical parameters, including the degree of lung damage, cytokine levels and liver enzymes in the circulation, indicate that the HP, besides facilitating robust and rapid binding of bacteria to E, can provide a degree of protection against the bacterial challenge.

TABLE I
Steady State PAO1 Distributions in Monkey 5D
Before mAb, After mAb and After HP Treatment^{a}

| | Particles^{b,d} | Particles^{b,d} | % Bound | CFU^{c,d} | CFU^{c,d} | % Bound |
|---|---|---|---|---|---|---|
| | SN | Pellet | (Particles)^{e} | SN | Pellet | (CFU)^{e} |
| Before mAb | 240,000 ± 100,000 | 220,000 ± 23,000 | 49 ± 9 | 220,000 ± 200,000 | 170,000 ± 34,000 | 51 ± 17 |
| (30-90 min) | | | | | | |
| After mAb, before | 97,000 ± 48,000 | 320,000 ± 31,000 | 77 ± 11 | 60,000 ± 22,000 | 350,000 ± 78,000 | 85 ± 7 |
| HP | | | | | | |
| (95-150 min) | | | | | | |
| After HP | 400 ± 300 | 690,000 ± 120,000 | 99.9 ± 0.1 | 1600 ± 1100 | 660,000 ± 27,000 | 99.8 ± 0.2 |
| (155-210 min) | | | | | | |

^{a}See also FIG. 5D legend for experimental details.
^{b}Particles/ml whole blood, determined by flow cytometry, see Materials and Methods, infra.
^{c}CFU/ml whole blood.
^{d}Average ± std. dev. of time points, n = 3 before mAb; n = 4 after mAb; n = 4 after HP.
^{e}% Bound (Particles) = 100 × (Pellet, Particles)/(Pellet, Particles + SN, Particles).
% Bound (CFU) = 100 × (Pellet, CFU)/(Pellet, CFU + SN, CFU).
% Bound is calculated for each individual time point and then averaged.

TABLE IIA
Clinical Parameters and CFU Assays for Selected Monkeys

| | Monkey | | | | | |
|---|---|---|---|---|---|---|
| | 6A^{a} | 6B^{a} | 7A^{b} | 7B^{b} | 7C^{c} | 7D^{c} |
| HP dose | Control | 125 ug/kg | Control | 107 ug/kg | Control | 216 ug/kg |
| PAO1 dose | 1.5 × 10⁹ | 1.8 × 10⁹ | 3 × 10⁹ | 2.7 × 10⁹ | 5 × 10⁹ | 6 × 10⁹ |
| (CFU/kg) | | | | | | |
| % PAO1 binding to | 32 | >99.9 | 98 | >99.9 | 89 | 99.7 |
| E^{d} | | | | | | |
| AST/ALT^{e} | 248/91 | 135/98 | 195/150 | 75/99 | 421/185 | 222/89 |
| HCT, Initial/Final | 37/29 | 33/28 | 31/26 | 40/22^{f} | 37/36 | 34/36 |
| CH50, Initial/Final | 120/180 | 369/415 | 204/170 | 363/342 | 450/290 | 453/344 |
| | CFU Assays on Organs (CFU/20 mg tissue)^{g} | | | | | |
| Liver | 300 | 50 | 200 | 105 | 3 | 5 |
| Spleen | 1.9 × 10⁴ | 330 | 65 | 130 | 370 | 30 |
| Lung | ND^{h} | ND | ND | ND | 1.6 × 10⁷ | 63 |
| Heart | 90 | ND | ND | ND | 8 × 10³ | ND |
| Kidney | 400 | ND | ND | ND | 800 | ND |

^{a}Cynomolgus monkeys, see FIG. 6. Monkey 6A was lethargic, depressed and withdrawn, and was euthanized at the 12 h mark. All other animals were euthanized at 24 h. Reciprocal IgG anti-PAO1 titers: 6A, 5; 6B, 17.
^{b}Cynomolgus monkeys. Reciprocal IgG anti-PAO1 titers: 7A, 50; 7B, >100.
^{c}Rhesus monkeys. Both 7C and 7D had reciprocal IgG anti-PAO1 titers >100. Monkey 7C was weak and flushed at the 24 h mark. Except for 6A and 7C, no other monkeys had observable symptoms at euthanasia.
^{d}Based on CFU determinations on whole blood and pelleted E, 60 minutes after start of bacterial infusion.
^{e}AST, aspartate aminotransferase, normal 32 ±8 U/L; ALT, alanine aminotransferase, normal 35 ± 7 U/L.
^{f}Leakage from arterial access site post-operatively. Further blood loss minimized by pressure wrap application.
^{g}CFU assays performed on tissue samples homogenized in 0.1% Triton in PBS at 5 gm tissue/20 ml PBS.
^{h}Not detectable.

TABLE IIB
Necropsy and pathology findings for selected monkeys

| PAO1 | | |
|---|---|---|
| dose | Control | HP-treated |
| 1.5 × 10⁹ | (6A) Diffuse congestion in lungs, liver | (6B) Diffuse |
| CFU/kg | and spleen. | congestion and |
| | | neutrophilia in |
| | | spleen and liver. |
| | | Lungs normal. |
| 3 × 10⁹ | (7A) Mild acute reactive hepatitis; mild | (7B) Mild acute |
| CFU/kg | to moderate acute congestion and edema | reactive splenitis; |
| | in the lungs; minimal lymphoid necrosis | no evidence of |
| | in the spleen. | tissue necrosis. |
| 6 × 10⁹ | (7C) Substantial consolidation in the | (7D) Lungs, healthy |
| CFU/kg | lungs, large numbers of hemorrhagic | and pink; two |
| | lesions throughout; mild focal acute | very small (pencil |
| | hemorrhagic pneumonia; minimal to | point) hemorrhagic |
| | mild reactive splenitis. | lesions; mild |
| | | leukocytosis, |
| | | small blood |
| | | vessels, liver; |
| | | minimal acute |
| | | reactive splenitis. |

TABLE III
Summary of Cytokine Levels after Infusion of PAO1^{a}

| | | | TNF-α | | | | | |
|---|---|---|---|---|---|---|---|---|
| Mky | | | at peak | | IL-6 at peak | | IL-1β at peak | |
| No./ | PAO1 dose | Inf. | | time | | time | | time |
| Treatment | CFU/kg/h | time h | ng/ml | min | ng/ml | min | ng/ml | min |
| 4C/CVF | 4 × 10⁸ | 2 | 50 ± 5 | 100 | 55 ± 18 | 180 | 0.13 ± 0.02 | 180 |
| 4D/CVF, HP | 8 × 10⁸ | 1 | 10 ± 4 | 120 | 19.1 ± 0.1 | 160 | 0.29 ± 0.01 | 160 |
| 6A/None | 1 × 10⁹ | 1.5 | 55 ± 7 | 150 | 131 ± 22 | 210 | 0.81 ± 0.10 | 150 |
| 6B/HP | 1.2 × 10⁹ | 1.5 | 8 ± 3 | 150 | 44 ± 8 | 210 | 0.075 ± 0.01 | 240 |
| 7A/None^{b} | 2 × 10⁹ | 1.5 | 41 ± 5 | 120 | 133 ± 6 | 210 | 1.58 ± 0.08 | 150 |
| 7B/HP^{b} | 1.8 × 10⁹ | 1.5 | 5 ± 1 | 135 | 66 ± 13 | 165 | 0.15 ± 0.02 | 150 |
| 5B/None^{c} | 1 × 10⁹ | 4 | 42 ± 13 | 120 | | | | |
| 5C/None | 3.5 × 10⁸ | 2 | 14 ± 2 | 90 | | | | |

^{a}Monkeys 4C, 4D, 6B, 7A, and 5B were treated with phenylephrine during the first 15 min of the experiments. Other monkeys were either not treated, or were treated after the TNF-α peak.
^{b}Values for TNF-α for 7A and 7B differ slightly from those shown in FIG. 7, which were determined as a time course in a different assay batch.
^{c}HP administered at 115 min; TNF-α was 24.5 ng/ml at 100 min.

TABLE IV
Relative Heteropolymer Binding and CR1 Levels on Selected Monkeys

| | ¹²⁵I goat anti-mouse IgG, cpm bound^{a} | | | | ¹²⁵I anti-CR1 mAb, cpm bound^{a,b} | | | |
|---|---|---|---|---|---|---|---|---|
| | | Post HP | | | | Post HP | | |
| Mky | Pre HP | <40 min | 3-4 hrs | 24 hrs | Pre HP | <40 min | 3-4 hrs | 24 hrs |
| 4A | 58 ± 2 | 2300 ± 200 | 1800 ± 200 | NA | 850 ± 50 | 600 ± 20 | 500 ± 20 | NA |
| 5A | 150 ± 10 | 1150 ± 50 | 800 ± 50 | NA | 2100 ± 100 | 1750 ± 100 | 1550 ± 100 | NA |
| 5D | 110 ± 30 | 550 ± 50 | 400 ± 20 | NA | 530 ± 10 | 420 ± 10 | 420 ± 10 | NA |
| 6B | 34 ± 3 | 550 ± 30 | 400 ± 10 | 300 ± 10 | 110 ± 5 | 97 ± 2 | 90 ± 5 | 91 ± 5 |
| 7D | 380 ± 10 | 1730 ± 20 | 1360 ± 20 | 650 ± 50 | 2560 ± 30 | 2250 ± 50 | 2100 ± 100 | 1300 ± 50 |

^{a}All results are normalized to cpm bound per 10⁶ E (mean ± SD).
^{b}Binding of ¹²⁵I anti-CR1 mAb to sheep E (lacking CR1) was less than 10% of the values observed for monkey E. Several different ¹²⁵I-labeled probes of different specific activities were used.

### 7. EXAMPLE 2

The following example demonstrates that a heteropolymer has the ability to remove a prototypical viral pathogen from the circulation of a primate.

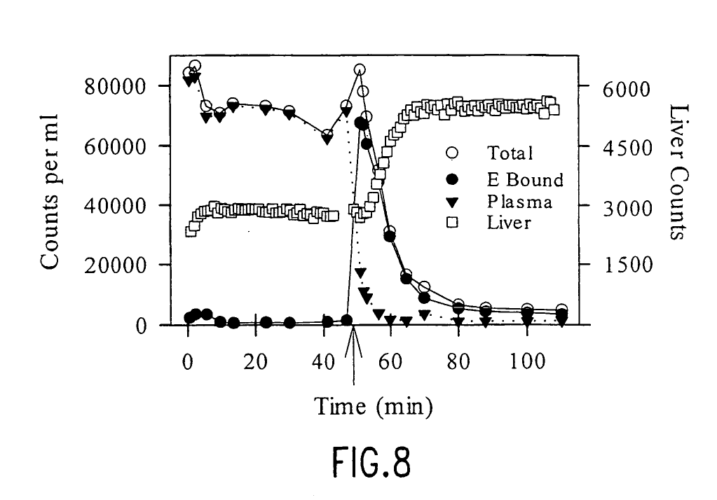

The ability of HP to remove a prototypical viral pathogen from the circulation was investigated. ¹³¹I-labeled bacteriophage ΦX174 was infused into the circulation of rhesus, cynomolgus and stump-tail macaques. The representative experiment illustrated in FIG. 8 shows that although there is some initial clearance, over the first 48 min most of the infused bacteriophage circulates freely in the plasma. After HP infusion, binding to E occurs rapidly. Soon after infusion of HP, a small amount of ¹³¹I ΦX174 returns to the circulation bound to E. We suggest that before HP infusion a fraction of the radiolabeled bacteriophage may be bound in the vasculature, and that the E-HP complex may serve to bind this adhered virus and return it to the bloodstream. Additional evidence that the HP can promote this recovery is presented below (see Dengue Virus Model).

After the ¹³¹I-labeled bacteriophage ΦX174 is bound to the E via HP, it is cleared from the circulation and localized principally to the liver, as demonstrated by Anger camera imaging (FIG. 8, right axis). In fact, the rate at which counts are cleared from the bloodstream appears to parallel the rate at which counts are taken up by the liver, and there is little, if any, release of counts into the plasma during this clearance process (FIG. 8). These results are also consistent with a concerted process in which acceptor cells in the liver remove and bind the substrate without allowing it to return to the plasma. Loss of E CR1 in these experiments was not detected, presumably because the studies were performed on monkeys with high E CR1 levels, and relatively small amounts of HP were infused. Additional tests on the animal the following day indicated that a very small fraction of the ¹³¹I remained in the liver, and counts were demonstrable in the bladder and the thyroid gland. The most reasonable explanation for this finding is that the material which cleared to the liver was subsequently phagocytosed and degraded, thus allowing free ¹³¹I⁻ to accumulate in the bladder and thyroid. Finally, infusion of 100 μg of ¹³¹I ΦX174 into the 9 kg animal would give a ΦX174 particle/E ratio of approximately 3 tol. Even at this relatively high challenge dose, the HP was able to facilitate close to quantitative binding of ΦX174 to the E (FIG. 8).

The anti-CR1 mAb component of the HP is essential for pathogen clearance. When anti-ΦX174 mAb 7B7 (used in the HP) alone was infused into a monkey, no immune adherence or clearance of the ΦX174 occurred. However, when an equimolar amount of HP (containing the same amount of mAb 7B7) was later infused into the animal, the ΦX174 was bound to the E and cleared. This result reinforces the utility of the anti-CR1 mAb as a surrogate for C3b. It binds with high avidity to E CR1, and as formulated in the HP, it can facilitate mAb-mediated binding of target pathogen to E under conditions of low concentration in which the anti-pathogen mAb by itself would not be sufficient to promote complement-mediated immune adherence.

### 8. EXAMPLE 3

The following example demonstrates that Fc receptors on macrophages play a key role in the clearance of immune complexes (IC).

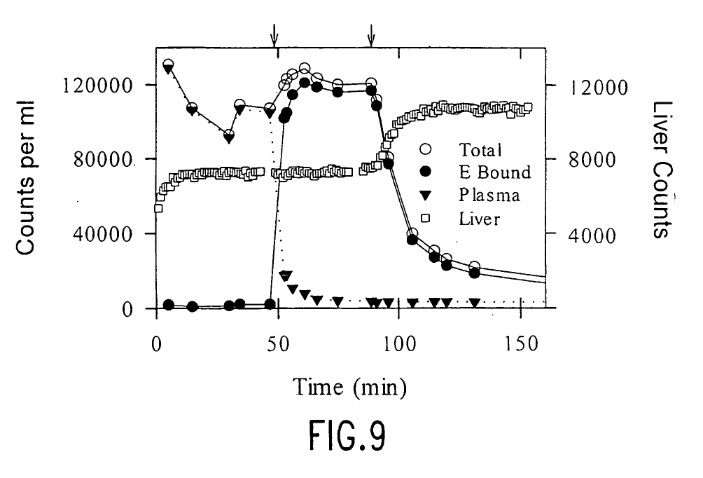

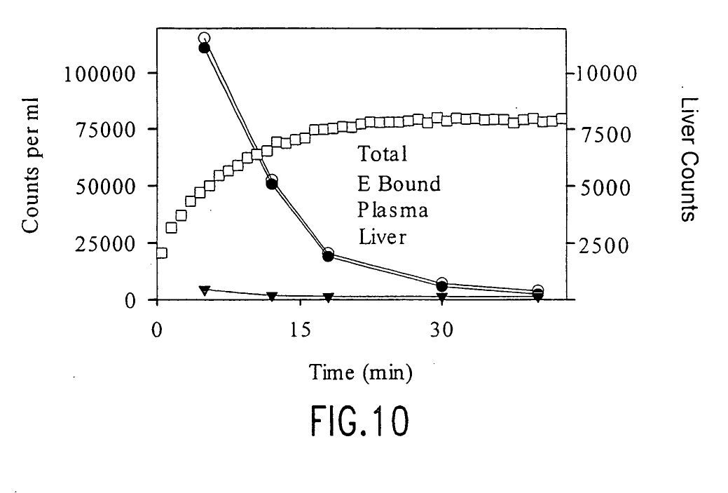

Fixed tissue macrophages have receptors specific for the Fc region of human IgG, and applicants believed that these receptors would play a key role in clearance of IC bound to E CR1 via either HP or complement opsonization. Therefore, HP lacking Fc regions were prepared by using IgG antibodies digested to produce Fab′ fragments. These modified HPs were then examined to determine how the modification affected handling of ¹³¹I-labeled bacteriophage ΦX174 in the circulation of a monkey (FIG. 9). The infused HP (7G9×7B7, Fab′ fragments, FIG. 9, left arrow at top axis, 48 min) were able to robustly bind the ΦX174 to E, but the ΦX174 was not removed from the E, thus providing evidence that indeed Fc recognition is important in the transfer reaction. Moreover, the slower clearance and greater stabilization of substrate bound to the E apparently enhanced recovery of bacteriophage from the vasculature, as evidenced by a larger number of counts returning to the circulation bound to E. ΦX174 is a multivalent particle, and accordingly it should be possible to promote clearance using whole IgG mAb 7B7 which would bind to additional epitopes on the ΦX174 not engaged by the Fab′-containing HP. In fact, infusion of mAb 7B7 at 88 min (FIG. 9, right arrow, top axis) promoted removal of E-bound material which was cleared and localized to the liver at a rate similar to that observed for the Fc-containing HP (FIG. 8).

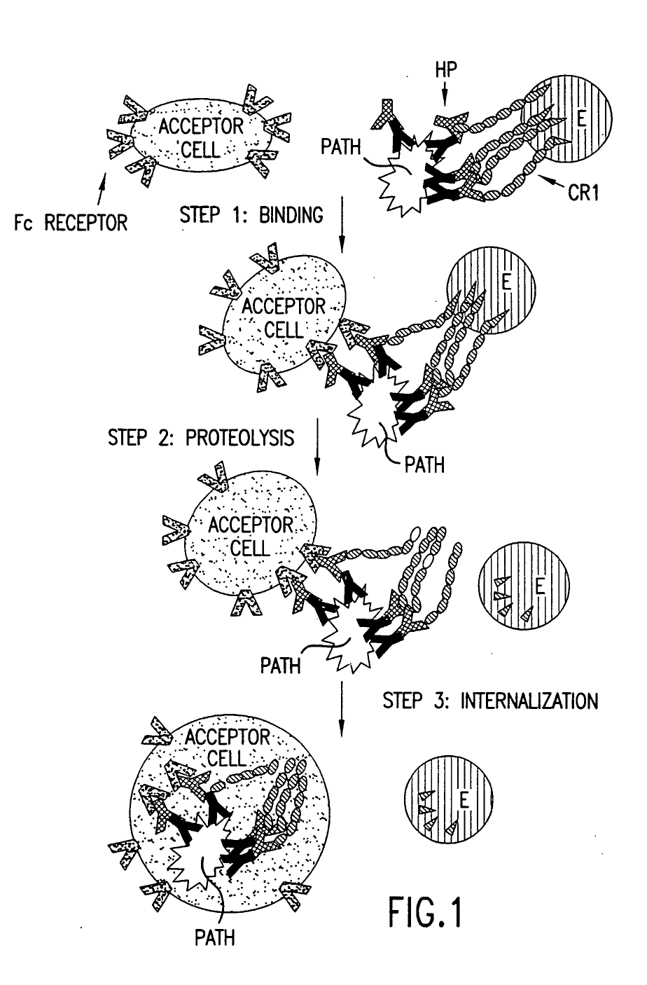

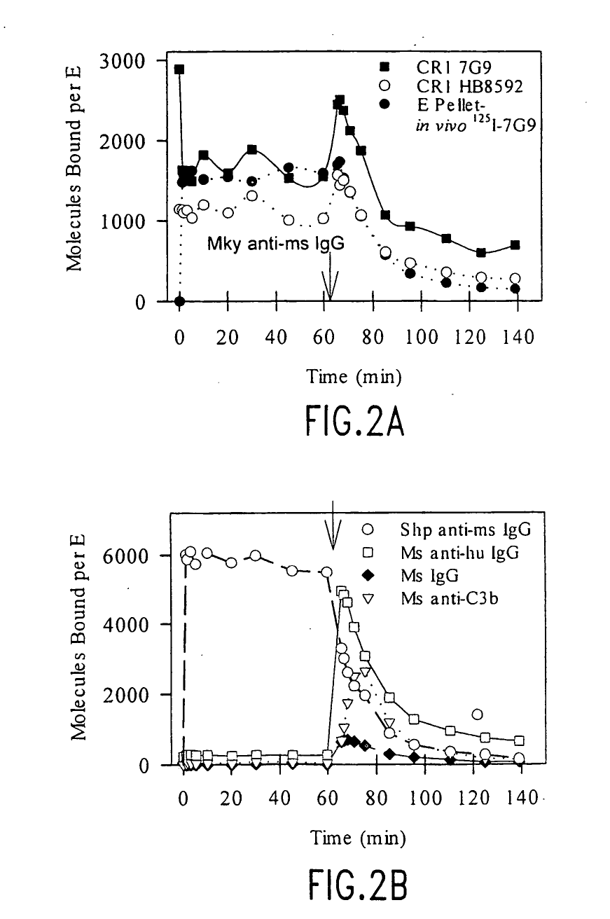

In order to evaluate the presumed similarity between HP-mediated clearance and natural C3b-mediated clearance of IC bound to E, another experiment was performed in a monkey that had been immunized with ΦX174. The animal had circulating IgG antibodies specific for ΦX174, and preliminary in vitro experiments confirmed that the sera of the animal supported immune adherence of the ΦX174 to monkey E. When the ¹³¹I-labeled bacteriophage ΦX174 was infused into this immunized monkey, the bacteriophage immediately bound to E and then was rapidly cleared from the circulation and localized to the liver with a kinetic profile comparable to that of HP-mediated clearance (FIG. 10). It is also important to note that, as observed in the HP-treated animal (FIG. 8), there was virtually no release of the E-bound IC back into the plasma. Presumably when the transfer reaction occurs there is weak association between the E and acceptor macrophages (FIG. 1) and thus, after CR1 is cleaved, the released material is taken up directly by the acceptor macrophages.

This experiment indicates that in immunized animals, multivalent antigens, such as particulate pathogens, will be rapidly bound to E by the immune adherence reaction and then removed from the circulation by phagocytic cell. FIGS. 8 and 9 reveal very similar patterns of clearance of E-bound ΦX174, although binding was accomplished either via the action of the HP or due to complement opsonization. We suggest that indeed the same mechanism of clearance was utilized in both cases. As visualized by Nelson, the “leukocyte,” in this case a liver macrophage, “scoured the erythrocyte surface,” engaged a specific locus, i.e., Fc containing IC, on the E and then cut CR1 and internalized the IC, and thus allowed it “to leave a normal appearing red blood corpuscle.” In further support of this mechanism, it is noted that in all of the in vivo clearance experiments, the mean corpuscular volume of E remained constant before and after HP-mediated clearance. Thus it appears that the transfer reaction is best thought of as a focused phagocytosis, a process which spares the E but removes the bound substrates.

The HP themselves constitute a very simple IC in that they are composed of two IgG molecules. E CR1 is organized in clusters and when large amounts of HP are bound to an E, it is likely that the clustered regions of CR1 will have a high local density of HP which should therefore be recognized as an IC. In fact, flow cytometry demonstrated that when amounts of HP sufficient to occupy more than 90% of E CR1 were infused into a monkey (2200 CR1/E), more than half of the HP were cleared from the E in 24 h, and accompanying this clearance was a loss of approximately 50% of CR1. Again, these results are most consistent with a concerted reaction in which the HP is cleared following proteolysis of CR1.

### 9. EXAMPLE 4

The following example demonstrates that a heteropolymer complex has the ability to remove a viral pathogen from the circulation of a primate.

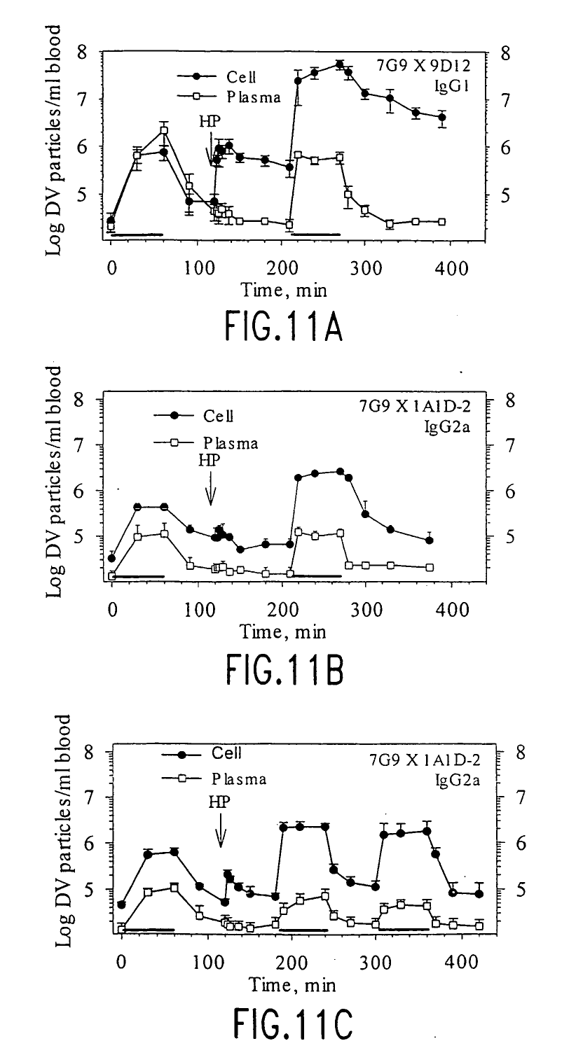

The high affinity IgG1 mouse mAb 9D12 specific for the surface E glycoprotein of dengue virus was used to prepare a suitable HP (Dr. Alan King of the Walter Reed Institute provided the monoclonal Ab). HP constructed with this mAb facilitated 85-90% binding of an attenuated strain of serotype 2 of dengue virus (DV) to human and monkey E. In all these binding and clearance experiments (see below) DV particles are quantitated in an RT-PCR assay. Acute viremia challenge studies in cynomolgus monkeys indicated that the HP was able to capture and bind DV when it was infused into the circulation of the monkeys. The dynamics of E binding and clearance of the infused DV revealed a very interesting story, as seen in an experiment which represents the general trends we observed (FIG. 11A). In the absence of HP, continuous infusion of DV led to a rather low steady-state level of DV in the bloodstream, and after the infusion ceased, the virus was rapidly cleared, although the site(s) for localization of cleared virus are not revealed in this experiment (FIG. 11A, first 120 min). During the infusion ˜50% of the infused virus was cell-associated, and we have found that this level of nonspecific binding is highly variable; in vitro experiments indicate 10-50% nonspecific binding of DV to both human and monkey E.

One h after the DV infusion was discontinued, the HP was infused. The amount of DV bound to the E increased dramatically within the first few min and was followed by a slow decline (FIG. 11A, 120-210 min). It is likely that this recovery represents DV which had adhered to vascular endothelial cells and was therefore accessible for ligation by the E-bound HP. The ability of the E-bound HP to capture DV is particularly well illustrated when the HP-treated monkey is challenged with a second continuous DV infusion (FIG. 11A, started at 210 min). The steady-state level of DV demonstrable in the circulation is increased almost 100-fold, and all of this DV is now bound to E. After the virus infusion is terminated the E-bound material is cleared, but at a relatively slow rate.

Although these experiments indicate that E-bound HP can bind and clear DV from the circulation, many questions remain to be addressed. In severe cases of secondary DV infections, dengue hemorrhagic fever (DHF) or dengue shock syndrome can result (DSS), and the level of virus in the bloodstream can exceed 10⁸ particles/ml. Thus, the E-HP system must be capable of binding large amount of DV. In addition, it is of critical importance that the E-bound DV be phagocytosed and destroyed after it is transferred to fixed tissue macrophages. It is likely that the fate of the transferred DV will depend upon the details of the process by which it is taken up by the acceptor macrophage, and therefore the role of Fc receptors in this reaction should be particularly important. In vitro and in vivo studies have indicated that the transfer reaction depends upon IC recognition by Fc receptors; based on the effects of specific mAbs in blocking the transfer reaction, it is anticipated that engagement of FcγRI in particular may insure the most efficient and rapid transfer. IgG2a mouse mAbs bind with the highest avidity to FcγRI, and thus DV bound to E with specific HP prepared with a mouse IgG2a anti-DV mAb would be cleared from the circulation more rapidly than HP prepared with the mouse IgG1 isotype.

Dr. John Roehrig of the CDC graciously provided IgG2a mAbs specific for the E-glycoprotein of DV (Virology 1998; 246:317-328), and HP (prepared according to the methods described in Section 6, infra) with these mAbs were able to bind large amounts of DV to both human E (not shown) and monkey E (Table V), thus addressing the first important consideration, that of the quantitative capacity of the system. Based on this data, the HP should be able to bind >10⁹ DV particles/ml to E in vivo. HP prepared with mAb 1A1D-2 was focused on, because this IgG2a mAb binds to the same epitope on the E glycoprotein of DV as mAb 9D1. In addition, and of considerable importance, in vitro calibration experiments also indicated that at comparable inputs the HP prepared with IgG2a mAb 1A1D-2 bound more DV to E than the HP prepared with mAb 9D12 (see Table V).

Two additional challenge studies were performed in monkeys to examine the potential of the new HP prepared with mAb 1A1D-2 to bind DV to E in the bloodstream and to facilitate its clearance. The conditions for the experiments depicted in FIGS. 11B, 11C (other than the third DV infusion in FIG. 11C) were otherwise quite similar, in terms of the amount of HP used and the level and timing of DV infusion, to those in FIG. 11A.

TABLE V
HP-mediated Binding of DV to Monkey E^{a}

| HP | % Bound^{b} |
|---|---|
| 7G9 × 9D12^{c} | 88 ± 4 |
| 7G9 × 1A1D-2^{d} | 96 ± 2 |
| 7G9 × 9A3D-8^{e} | 94 ± 1 |
| no HP | 26 ± 2 |
| 7G9 | 26 ± 2 |

^{a}5 × 10⁷ monkey E were franked with excess HP, washed and incubated with 2.5 × 10⁷ DV particles, in a total volume of 15 μl.
^{b}mean ± SD, n = 4.
^{c}mAb 7G9 is IgG2a (Ferguson et al., 1995, Arthritis Rheum 38:190), and mAb 9D12 is IgG1 (Gentry et at., 1982, Am J. Trop. Med. Hyg 31:548)
^{d}IgG2a (Roehrig et al., 1998, Virology 246:317), same specificity as mAb 9D12.
^{e}IgG2a (Roehrig et al., 1998, Virology 246:317).

Comparison of FIG. 11A with FIGS. 11B and 11C indicates that before HP infusion, the pattern of DV presentation in the bloodstream was quite similar in all three monkeys. However, several new trends are evident after monkeys 11B and 11C are treated with the HP prepared with the 1A1D-2 IgG2a mAb. First, after HP infusion less DV could be recovered in the bloodstream, but again the recovered DV was bound to E. Second, after HP treatment, the steady state plateau achieved upon re-challenge with DV was considerably reduced for monkeys 11B and 11C. Finally, after the DV infusion was terminated, levels of DV in the circulation decreased quite rapidly and approached the limit of detection. Even in monkey 11C, which was challenged twice after HP treatment, the same trends are manifest.

One possible explanation for these findings is that the HP prepared with mAb 1A1 D-2 binds DV less well or perhaps at a different epitope than the HP prepared with mAb 9D12. However, in vitro calibrations (Table V) indicate that the new HP binds DV at least as well as the 9D12 HP, and the fact that the same epitope is recognized would argue that the potential of the new HP to recognize, bind and recover DV from the vasculature should certainly be comparable to that of the 9D12 HP. The results illustrated in FIGS. 11B and 11C are consistent with the supposition that the HP prepared with the IgG2a anti-DV mAb facilitates faster clearance. That is, after HP infusion, DV was recovered bound to E, but the steady state level of DV bound to E was quite low, because it was presumably cleared rapidly. Upon challenge of the animals with DV after HP treatment, the steady state level of DV bound to E during continuous virus infusion was lower, again due to its more rapid removal from the E. Finally, after the DV infusion was terminated, as expected residual virus bound to E was cleared rapidly.

The consequences of secondary infections with different serotypes of DV are well known, and there is good reason to believe that, especially at lower antibody levels, binding of host antibodies to DV may enhance infection of monocyte/macrophages and promote the most virulent forms of the disease including DHF and DSS. An important question concerns the role of Fc receptors in this reaction. Based on the in vivo and in vitro studies with ΦX174, we suggest that if a virus in the bloodstream is opsonized with a sufficient number of HP and bound to E CR1, then, as discussed above, processing during the transfer reaction will result in its phagocytosis and destruction. That is, the processing and degradation of the E-bound HP-virus complex should follow the natural pathway for processing of opsonized multivalent pathogens bound to E first described by Nelson.

The present invention is not to be limited in scope by the exemplified embodiments, which are intended as illustrations of single aspects of the invention. Indeed, various modifications of the invention in addition to those shown and described herein will become apparent to those skilled in the art from the foregoing description and accompanying drawings. Such modifications are intended to fall within the scope of the appended claims.

All patents and publications cited herein are incorporated by reference in their entirety.

1. A heteropolymer complex comprising a first monoclonal antibody specific for a C3b-like receptor of a mammal chemically crosslinked (covalently linked) to a second monoclonal antibody, in which the isotype of at least the second monoclonal is the isotype having the highest known affinity for the Fc receptor in said mammal.

2. The complex of claim 1 in which the first monoclonal antibody is specific for the complement receptor on a primate erythrocyte.

3. The complex of claim 2 in which the primate erythrocyte is a human erythrocyte.

4. A heteropolymer complex, which complex comprises a first monoclonal antibody specific complement receptor CR1 expressed on a human erythrocyte chemically crosslinked to a second monoclonal antibody, in which the isotype of at least the second monoclonal antibody is human IgG1 or human IgG3.

5. The complex of claim 4, in which the second monoclonal antibody is a human, humanized or chimeric antibody.

6. The complex of claim 4 in which the first monoclonal antibody is a human, humanized or chimeric antibody.

7. The complex of claim 4 in which the isotype of the first monoclonal antibody is human IgG1 or human IgG3.

8. The complex of claim 4 in which the first monoclonal antibody is selected from the group consisting of 7G9, 1B4, 3D9, E-11, 57F, YZ1, and HB8592.

9. A heteropolymer cocktail composition comprising at least two heteropolymer complexes, in which at least one complex comprises a first monoclonal antibody specific for a C3b-like receptor of a mammal chemically crosslinked to a second monoclonal antibody, in which the isotype of at least the second monoclonal antibody is the isotype having the highest known affinity for the Fc receptor in said mammal.

10. The complex of claim 4 in which the second monoclonal antibody specifically binds a viral antigen.

11. The complex of claim 10 in which the viral antigen is an antigen of a retrovirus, a herpes virus, an arenavirus, a paramyxovirus, an adenovirus, a bunyavirus, a cornavirus, a filovirus, a flavivirus, a hepadnavirus, an orthomyovirus, a papovavirus, a picornavirus, a poxvirus, a reovirus, a togavirus, or a rhabdovirus.

12. The complex of claim 10 in which the viral antigen is selected from the group consisting of HIV gp120, influenza neuramimidase, influenza hemagglutinin, and RSV F glycoprotein.

13. The complex of claim 4 in which the second monoclonal antibody specifically binds a microbial antigen.

14. The complex of claim 13 in which the microbial antigen is lipopolysaccharide.

15. The complex of claim 13 in which the microbial antigen is an antigen of Streptococcus sp., Streptococcus sp., Neisseria sp., Corynebacterium sp., Clostridium sp., Haemophilus sp., Klebsiella sp., Staphylococcus sp., Vibrio sp., Escherichia sp., Pseudomonas sp., Campylobacter (Vibrio) sp., Aeromonas sp., Bacillus sp., Edwardsiella sp., Yersinia sp., Shigella sp., Salmonella sp., Treponema sp., Borrelia sp., Leptospira sp., Mycobacterium sp., Toxoplasma sp., Pneumocystis sp., Francisella sp., Brucella sp., Mycoplasma sp., Rickettsia sp., Chlamydia sp., or Helicobacter sp.

16. The complex of claim 4 in which the second monoclonal antibody specifically binds a cancer cell-specific antigen.

17. The complex of claim 16 in which the cancer cell-specific antigen is selected from the group comprising CD20, Her-2, and PSMA.

18. A method for immune clearance of an antigen comprising administering to a mammal an effective amount of a heteropolymer complex according to any of claims 1-17.

19. A method for immune clearance of an antigen comprising administering to a mammal an effective amount of a heteropolymer complex cocktail according to claim 9.

20. A method for immune clearance of an antigen comprising administering to a mammal an effective amount of franked cells expressing a C3b-like receptor bound to a heteropolymer complex, said complex comprising a first monoclonal antibody specific for the C3b-like receptor of said mammal chemically crosslinked to a second monoclonal antibody, in which the isotype of at least the second monoclonal antibody is the isotype having the highest known affinity for the Fc receptor in said mammal.

21. A method of detecting the presence of an antigen in a mammal, said method comprising contacting a sample obtained from the mammal containing cells expressing a C3b-like receptor with a heteropolymer complex according to any of claims 1-17; and detecting binding of the antigen in the sample.

22. A method for treating or preventing viral infection or microbial infection in a mammal comprising administering to said mammal an effective amount of a heteropolymer complex according to any of claim 1-17.

23. The method of claim 22 in which the viral infection is caused by a retrovirus, a herpes virus, an arenavirus, a paramyxovirus, an adenovirus, a bunyavirus, a cornavirus, a filovirus, a flavivirus, a hepadnavirus, an orthomyovirus, a papovavirus, a picornavirus, a poxvirus, a reovirus, a togavirus, or a rhabdovirus.

24. The method of claim 22 in which the microbial infection is a yeast infection, fungal infection, protozoan infection or bacterial infection.

25. The method of claim 24 in which the bacterial infection is caused by Streptococcus sp., Streptococcus sp., Neisseria sp., Corynebacterium sp., Clostridium sp., Haemophilus sp., Klebsiella sp., Staphylococcus sp., Vibrio sp., Escherichia sp., Pseudomonas sp., Campylobacter (Vibrio) sp., Aeromonas sp., Bacillus sp., Edwardsiella sp., Yersinia sp., Shigella sp., Salmonella sp., Treponema sp., Borrelia sp., Leptospira sp., Mycobacterium sp., Toxoplasma sp., Pneumocystis sp., Francisella sp., Brucella sp., Mycoplasma sp., Rickettsia sp., Chlamydia sp., or Helicobacter sp.

26. The method of claim 22 in which the complex is administered intravenously.

27. The method of claim 22, in which the complex is administered intravenously to a human in an amount of 1-10 mg.

28. The method of claim 22 in which the microbial antigen is lipopolysaccharide.

29. A method for treating or preventing septic shock in a mammal comprising administering to said mammal an effective amount of a heteropolymer complex according to any of claims 1-17.

30. The method of claim 29 in which the complex is administered intravenously.

31. The method of claim 29, in which the complex is administered intravenously to a human in an amount of 1-10 mg.

32. The method of claim 31 in which the human is immunocompromised, immunodeficient, elderly, suffering from burns, or an infant.

33. A method for treating cancer in a mammal comprising administering to said mammal an effective amount of a heteropolymer complex according to any of claims 1-17.

34. The method of claim 33 in which the complex is administered intravenously.

35. The method of claim 33, in which the complex is administered intravenously to a human in an amount of 1-10 mg.
